(19) United States
(12) Patent Application Publication
(10) Pub. No.: US 2005/0283042 A1
(43) Pub. Date: Dec. 22, 2005

(54) Cardiac harness having radiopaque coating and method of use

(76) Inventors: Steve Meyer, Oakland, CA (US); Lilip Lau, Los Altos, CA (US)

Correspondence Address: FULWIDER PATTON LEE & UTECHT, LLP;HOWARD HUGHES CENTER, 6060 CENTER DRIVE, TENTH FLOOR, LOS ANGELES, CA, 90045, US

(21) Appl. No.: 11/190,500

(22) Filed: Jul. 26, 2005

Related U.S. Application Data
(63) Continuation-in-part of application No. 10/811,245, filed on Mar. 25, 2004.
(60) Provisional application No. 60/458,991, filed on Mar. 28, 2003.

(51) Int. Cl. A61F 13/00; A61F 2/00

(52) U.S. Cl. 600/37

(57) ABSTRACT

A cardiac harness is configured to fit at least a portion of a patient's heart and at least a portion of the cardiac harness includes a radiopaque coating. The radiopaque coating is formed from a polymer loaded with a radiopaque material such as barium sulfate. The radiopaque material also includes a radiopaque band or a radiopaque coating formed by sputter coating or the like. The radiopaque material is visible under fluoroscopy, MRI, or echocardiography.

This is a continuation-in-part of U.S. Ser. No. 10/811,245 filed Mar. 25, 2004, which depends for priority upon U.S. Provisional Patent Application No. 60/458,991, filed Mar. 28, 2003, the entirety of each of which is herein incorporated by reference.

### BACKGROUND OF THE INVENTION

The present invention relates to a device for treating heart failure. More specifically, the invention relates to a cardiac harness configured to be fit around at least a portion of a patient's heart. The cardiac harness is configured to be radiopaque for enhanced visualization under fluoroscopy. The cardiac harness includes an arrangement that provides no electrical continuity circumferentially about the harness, and therefore allows an electric current to pass through the heart unimpeded. In a situation where a defibrillating electrode positioned inside the right ventricle of a patient is connected to an implantable cardiac defibrillator (“ICD”), the arrangement of the cardiac harness will allow the defibrillating current to pass from the electrode inside of the heart to the ICD without the current being conducted around the heart through the harness. Also, if defibrillator paddles are applied to a harness that is placed on a patient's heart, the electric current created between the paddles would pass through the heart instead of being conducted around the heart through the harness.

Congestive heart failure (“CHF”) is characterized by the failure of the heart to pump blood at sufficient flow rates to meet the metabolic demand of tissues, especially the demand for oxygen. One characteristic of CHF is remodeling of at least portions of a patient's heart. Remodeling involves physical change to the size, shape and thickness of the heart wall. For example, a damaged left ventricle may have some localized thinning and stretching of a portion of the myocardium. The thinned portion of the myocardium often is functionally impaired, and other portions of the myocardium attempt to stroke volume of the ventricle is maintained notwithstanding the impaired zone of the myocardium. Such expansion may cause the left ventricle to assume a somewhat spherical shape.

Congestive heart failure (“CHF”) is characterized by the failure of the heart to pump blood at sufficient flow rates to meet the metabolic demand of tissues, especially the demand for oxygen. One characteristic of CHF is remodeling of at least portions of a patient's heart. Remodeling involves physical change to the size, shape and thickness of the heart wall. For example, a damaged left ventricle may have some localized thinning and stretching of a portion of the myocardium. The thinned portion of the myocardium often is functionally impaired, and other portions of the myocardium attempt to compensate. As a result, the other portions of the myocardium may expand so that the stroke volume of the ventricle is maintained notwithstanding the impaired zone of the myocardium. Such expansion may cause the left ventricle to assume a somewhat spherical shape.

Cardiac remodeling often subjects the heart wall to increased wall tension or stress, which further impairs the heart's functional performance. Often, the heart wall will dilate further in order to compensate for the impairment caused by such increased stress. Thus, a cycle can result, in which dilation leads to further dilation and greater functional impairment.

One of the problems encountered with prior prosthetic girdles used to treat congestive heart failure is that they typically are made from materials that are virtually transparent under x-ray or fluoroscopy. For example, prosthetic socks or jackets are made from a polymer material that cannot be visualized under standard fluoroscopy conditions.

Accordingly, the present invention includes embodiments of a cardiac harness that is visible under fluoroscopy, x-ray, and other visualization means, especially useful during minimally invasive delivery of the cardiac harness.

### SUMMARY OF THE INVENTION

In accordance with the present invention, a cardiac harness is configured to fit at least a portion of a patient's heart and includes a conductive material that is coated with a dielectric coating to electrically insulate the conductive material from an electric current that may be applied to the heart. In one embodiment, the conductive material is entirely coated with the dielectric coating so that the entire harness is electrically insulated. The conductive material can be a metallic wire, and preferably one having a shape memory property, and the dielectric coating can include Parylene™, silicone rubber, urethane, polytetrafluoroethylene, or an elastomer. In certain embodiments, the conductive material comprises a wire that formed into a plurality of hinge members.

In keeping with the invention, the cardiac harness was configured to fit at least a portion of a patient's heart and is coated with a dielectric coating or other polymer, preferably to electrically insulate the conductive material from an electric current that may be applied to the heart. In this embodiment, the dielectric coating or other polymer material is loaded with a radiopaque substance that will enhance the visualization of the cardiac harness when viewed under fluoroscopy or other x-ray devices. In one embodiment, the polymer is a silicone rubber that is loaded with a barium sulfate compound in order to increase the radiopacity of the silicone rubber so that it is highly visible under fluoroscopy. Other polymers that can be loaded with a radiopaque substance include Parylene™, urethane, polytetrafluoroethylene or an elastomer.

In another embodiment, a cardiac harness is configured to fit at least a portion of a patient's heart and includes a conductive material that is coated with a dielectric coating or other polymer, preferably to electrically insulate the conductive material from the electric current that may be applied to the heart. In this embodiment, the dielectric coating can include Parylene™, silicone rubber, urethane, polytetrafluoroethylene or an elastomer. Further, in order to enhance the radiopacity of the cardiac harness, bands of highly radiopaque material are attached to the cardiac harness, at various locations, which bands are easily visible under fluoroscopy. The bands can be formed of highly radiopaque materials such as gold, silver, platinum iridium, and the like. The bands typically are biocompatible and can be crimped or otherwise attached to the cardiac harness, preferably on straight sections of the cardiac harness so as not to interfere with the bending of the cardiac harness during delivery and during use. Highly radiopaque bands can be attached to the cardiac harness by crimping, swaging, or bonding onto the wire material of the cardiac harness. In one embodiment, the bands can be formed on the cardiac harness by plating, plasma deposition, or other similar processes known in the art.

Also in accordance with the present invention, a method of manufacturing a cardiac harness includes providing a metallic wire, covering the wire with a dielectric material, and forming the wire into a plurality of spring members. In one embodiment, the dielectric material is in the form of a tube that is slid over the metallic wire, such that the wire is insulated by the dielectric material. In this embodiment, the dielectric material is loaded with a radiopaque substance, such as barium sulfate, so that the dielectric material not only insulates the cardiac harness from electrical impulses, but also is highly radiopaque under fluoroscopy. Any excess dielectric material is removed from the wire so that the shape of the dielectric material generally follows the shape of the spring members.

In another embodiment, a method of manufacturing a cardiac harness includes, etching at least one spring member out of a flat sheet of conductive material. In this embodiment the etched spring member is then coated with a dielectric material, such that the etched spring member is insulated by the dielectric material, and any excess dielectric material is removed from the etched spring member so that the shape of the dielectric material generally follows the shape of the spring members. In this embodiment, the dielectric material is loaded with a radiopaque substance, such as barium sulfate, so that the dielectric material not only insulates the cardiac harness from electrical impulses, but also is highly radiopaque under fluoroscopy.

### BRIEF DESCRIPTION OF THE DRAWINGS

FIG. 1 depicts a schematic view of a heart with a prior art cardiac harness placed thereon.

FIGS. 2A-2B depict a spring hinge of a prior art cardiac harness in a relaxed position and under tension.

FIG. 3 depicts a prior art cardiac harness that has been cut out of a flat sheet of material.

FIG. 4 depicts the prior art cardiac harness of FIG. 3 formed into a shape configured to fit about a heart.

FIG. 5A depicts a flattened view of one embodiment of the cardiac harness of the invention showing two panels connected to two electrodes.

FIG. 5B depicts a cross-sectional view of an electrode.

FIG. 5C depicts a cross-sectional view of an electrode.

FIG. 5D depicts a cross-sectional view of an electrode.

FIG. 5E depicts a portion of an undulating strand having radiopaque bands.

FIG. 5F depicts a portion of an undulating strand having a portion coated with a radiopaque material.

FIG. 6A depicts a cross-sectional view of an undulating strand or ring.

FIG. 6B depicts a cross-sectional view of an undulating strand or ring.

FIG. 6C depicts a cross-sectional view of an undulating strand or ring.

FIG. 7A depicts an enlarged plan view of a cardiac harness showing three electrodes separating three panels, with the far side panel not shown for clarity.

FIG. 7A depicts an enlarged plan view of a cardiac harness showing three electrodes separating three panels, with the far side panel not shown for clarity.

FIG. 7B depicts an enlarged partial plan view of the cardiac harness of FIG. 7A showing an electrode partially covered with a dielectric material which also serves to attach the panels to the electrode.

FIG. 8 depicts a plan view of one embodiment of a cardiac harness having panels separated by and attached to flexible coils.

FIG. 9 depicts a flattened plan view of a cardiac harness similar to that of FIG. 8 but with fewer panels and coils.

FIG. 10 depicts a plan view of one embodiment of a cardiac harness having panels separated by and attached to flexible coils.

FIG. 11 depicts a plan view of a cardiac harness similar to that shown in FIG. 10 mounted on the epicardial surface of the heart.

FIG. 12 depicts a plan view of a portion of an undulating strand having radiopaque bands.

FIG. 13 depicts a plan view of a portion of a cardiac harness showing panels separated by radiopaque connectors.

FIG. 14 depicts a perspective view of another embodiment of a cardiac harness having a plurality of rings covered with a dielectric material and disposed longitudinally adjacent to one another.

FIG. 15 depicts a partial cross-sectional view of opposite ends of each ring attached to one another by a connective junction.

FIG. 16 depicts an unattached elongated strand or series of spring elements.

FIG. 17 depicts a perspective view of another embodiment of a cardiac harness having a plurality of rings covered with a dielectric material and adjacent rings are interconnected with nonconductive connectors.

FIG. 18 depicts a connective junction joining opposite ends of a ring with a dielectric material.

FIG. 19 depicts an elongated strand having a harness portion and a leader portion.

FIGS. 20 and 21 depict a schematic view of a wire being covered with a silicone rubber tube.

FIG. 22 depicts a side view of an introducer for delivering the cardiac harness through minimally invasive procedures.

FIG. 23 depicts a perspective end view of a dilator with the cardiac harness releasably positioned therein.

FIG. 24 depicts a schematic cross-sectional view of a human thorax with the cardiac harness system being delivered by a delivery device inserted through an intercostal space and contacting the heart.

FIG. 25 depicts an end view of the introducer with the cardiac harness releasably positioned therein.

FIG. 26 depicts a plan view of the heart with a suction device releasably attached to the apex of the heart.

FIG. 27 depicts a plan view of the heart with the suction device attached to the apex and the introducer positioned to deliver the cardiac harness over the heart.

FIG. 28A depicts a plan view of the cardiac harness being deployed from the introducer onto the epicardial surface of the heart.

FIG. 28B depicts a plan view of the cardiac harness being deployed where portions of the harness have increased radiopacity.

FIG. 29A depicts a plan view of the heart with the cardiac harness being deployed from the introducer onto the epicardial surface of the heart.

FIG. 29B depicts a plan view of the cardiac harness being deployed where portions of the harness have increased radiopacity.

FIG. 30A depicts a plan view of the heart with the cardiac harness having electrodes attached thereto, surrounding a portion of the heart.

FIG. 30B depicts a plan view of the cardiac harness being deployed where portions of the harness have increased radiopacity.

FIG. 31A depicts a schematic view of the cardiac harness assembly mounted on the human heart together with leads and an ICD for use in defibrillation or pacing.

FIG. 31B depicts a plan view of the cardiac harness being deployed where portions of the harness have increased radiopacity.

### DETAILED DESCRIPTION OF THE PREFERRED EMBODIMENTS

The present invention relates to a method and apparatus for treating heart failure. As discussed in Applicants' co-owned and co-assigned patent entitled “Expandable Cardiac Harness For Treating Congestive Heart Failure”, U.S. Pat. No. 6,702,752, which was filed on Aug. 8, 2000, the entirety of which is hereby expressly incorporated by reference herein, it is anticipated that remodeling of a diseased heart can be resisted or even reversed by alleviating the wall stresses in such a heart. The present disclosure discusses certain embodiments and methods for supporting the cardiac wall. Additional embodiments and aspects are also discussed in Applicants' co-pending applications entitled “Heart Failure Treatment Device and Method,” Ser. No. 10/287,723, filed Oct. 31, 2002, “Method and Apparatus for Supporting a Heart,” Ser. No. 10/338,934, filed Jan. 7, 2003, “Cardiac Harness,” Ser. No. 10/656,722, filed Sep. 5, 2003, and Applicant's co-owned and co-assigned patent entitled “Device for Treating Heart Failure,” U.S. Pat. No. 6,723,041, the entirety of each of which are hereby expressly incorporated by reference.

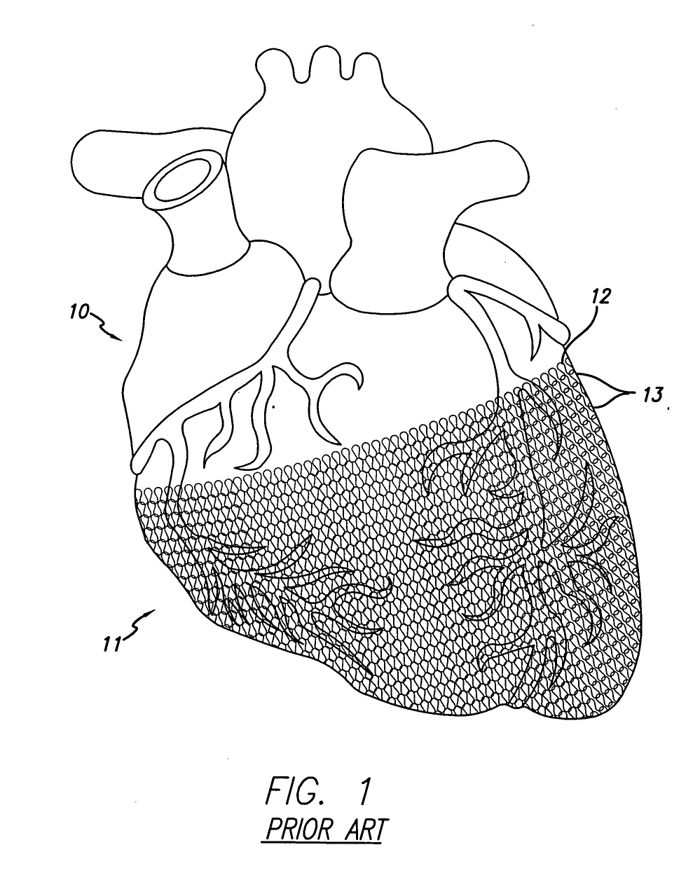

FIG. 1 illustrates a mammalian heart 10 having a prior art cardiac wall stress reduction device in the form of a harness 11 applied to it. The cardiac harness has a series of hinges or spring elements 12 that circumscribe the heart and, collectively, apply a mild compressive force on the heart so as to alleviate wall stresses.

The term “cardiac harness” as used herein is a broad term that refers to a device fit onto a patient's heart to apply a compressive force on the heart during at least a portion of the cardiac cycle.

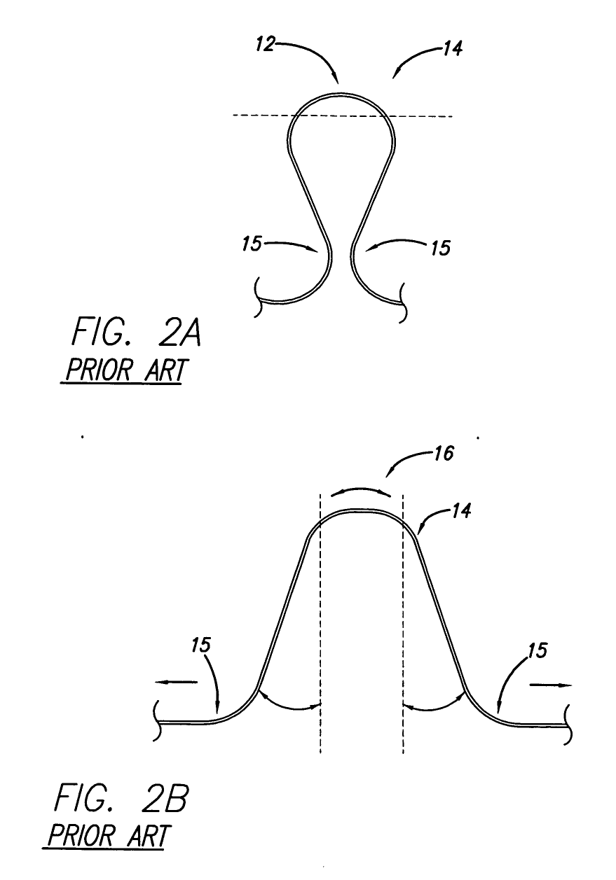

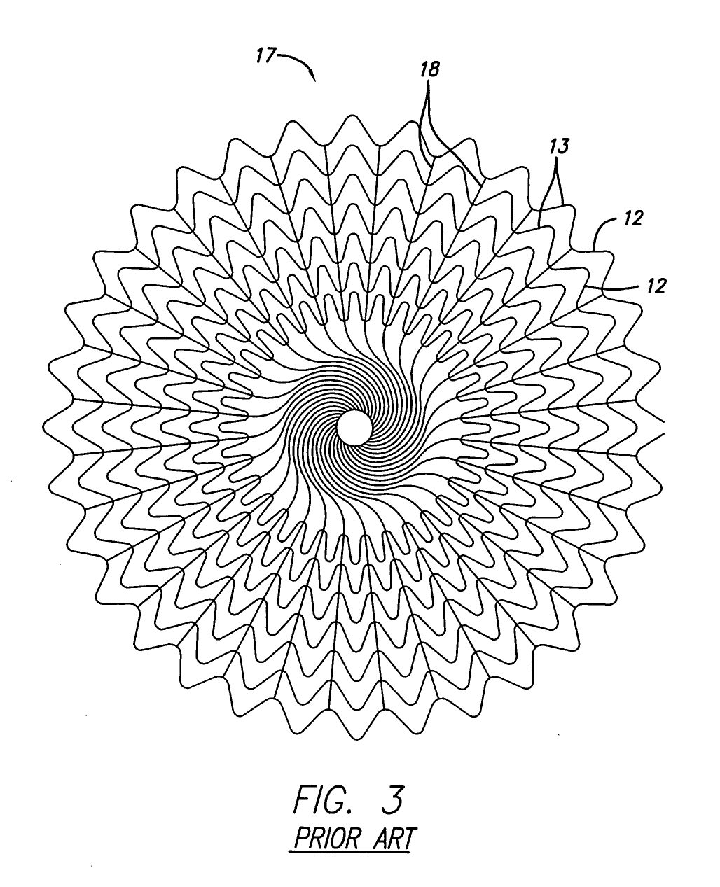

The cardiac harness 11 illustrated in FIG. 1 has at least one undulating strand 13 having a series of spring elements 12 referred to as hinges or spring hinges that are configured to deform as the heart 10 expands during filling. Each hinge provides substantially unidirectional elasticity, in that it acts in one direction and does not provide much elasticity in the direction perpendicular to that direction. For example, FIG. 2A shows one embodiment of a prior art hinge member at rest. The hinge member has a central portion 14 and a pair of arms 15. As the arms are pulled, as shown in FIG. 2B, a bending moment 16 is imposed on the central portion. The bending moment urges the hinge member back to its relaxed condition. Note that a typical strand comprises a series of such hinges, and that the hinges are adapted to elastically expand and retract in the direction of the strand.

In the harness illustrated in FIG. 1, the strands 13 of spring elements 12 are constructed of extruded wire that is deformed to form the spring elements.

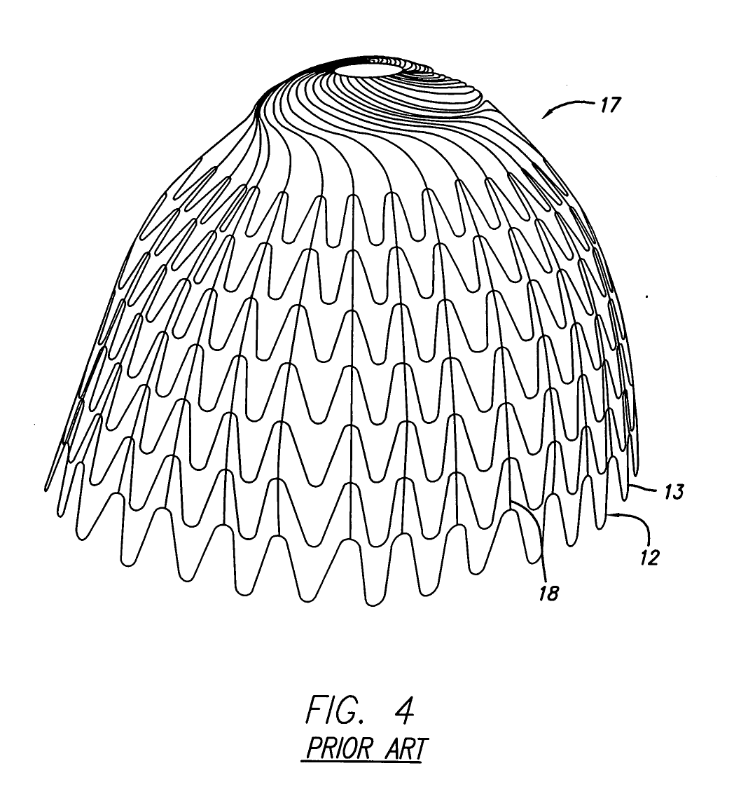

FIGS. 3 and 4 illustrate another prior art cardiac harness 17, shown at two points during manufacture of such a harness. The harness is first formed from a relatively thin, flat sheet of material. Any method can be used to form the harness from the flat sheet. For example, in one embodiment, the harness is photochemically etched from the material; in another embodiment, the harness is laser-cut from the thin sheet of material. The harness shown in FIGS. 3 and 4 has been etched from a thin sheet of Nitinol, which is a superelastic material that also exhibits shape memory properties. The flat sheet of material is draped over a form, die or the like, and is formed to generally take on the shape of at least a portion of a heart.

With further reference to FIGS. 1 and 4, the cardiac harnesses 11, 17 have a base portion, which is sized and configured to generally engage and fit onto a base region of a patient's heart; an apex portion, which is sized and shaped so as to generally engage and fit on an apex region of a patient's heart; and a medial portion between the base and apex portions.

In the harness shown in FIGS. 3 and 4, the harness 17 has strands or rows 13 of undulating wire. As discussed above, the undulations have hinges/spring elements 12 which are elastically bendable in a desired direction. Some of the strands are connected to each other by interconnecting elements 18. The interconnecting elements help maintain the position of the strands relative to one another. Preferably the interconnecting elements allow some relative movement between adjacent strands.

The undulating spring elements 12 exert a force in resistance to expansion of the heart 10. Collectively, the force exerted by the spring elements tends toward compressing the heart, thus alleviating wall stresses in the heart as the heart expands. Accordingly, the harness helps to decrease the workload of the heart, enabling the heart to more effectively pump blood through the patient's body and enabling the heart an opportunity to heal itself. It should be understood that several arrangements and configurations of spring members can be used to create a mildly compressive force on the heart to reduce wall stresses. For example, spring members can be disposed over only a portion of the circumference of the heart or the spring members can cover a substantial portion of the heart.

As the heart expands and contracts during diastole and systole, the contractile cells of the myocardium expand and contract. In a diseased heart, the myocardium may expand such that the cells are distressed and lose at least some contractility. Distressed cells are less able to deal with the stresses of expansion and contraction. As such, the effectiveness of heart pumping reduces. Each spring member of the above cardiac harness is configured so that as the heart expands during diastole the spring members correspondingly will expand, thus storing expansion forces as bending energy in the spring. As such, the stress load on the myocardium is partially relieved by the harness. This reduction in stress helps the myocardium cells to remain healthy and/or regain health. As the heart contracts during systole, the disclosed prior art cardiac harness applies a moderate compressive force as the hinge or spring elements release the bending energy developed during expansion allowing the cardiac harness to follow the heart as it contracts and to apply contractile forces as well.

Other structural configurations for cardiac harnesses exist, however, but all have drawbacks and do not function optimally to treat CHF and other related diseases or failures. The present invention provides a novel approach to treat CHF and includes a cardiac harness that not only is highly visible under fluoroscopy but also prevents electrical continuity circumferentially about the harness.

The present invention is directed to a cardiac harness that is fitted around at least a portion of the patient's heart to apply a compressive force on the heart during diastole and systole, and the harness includes a material that enhances visibility under fluoroscopy. Preferably, the harness also includes a configuration that does not allow electrical continuity circumferentially about the harness. Therefore, if an electric current created by a defibrillation device (external defibrillator, automatic external defibrillator, or ICD) is applied to a patient who has a harness placed on their heart, the electric current will pass through the heart instead of being conducted around the heart through the harness. In one embodiment, the cardiac harness can be formed with a conductive material that is coated with a dielectric coating to prevent electrical continuity circumferentially about the harness. The dielectric coating over the conductive material of the harness preferably is loaded with a material that is highly visible under fluoroscopy or other known visualization methods used in operating rooms, cath labs, and the like. In another embodiment, various configurations of panels are connected together with a dielectric material, thereby electrically isolating each panel from one another in the harness. Similarly, the dielectric material which electrically isolates each panel from one another in the harness, also can be loaded with a material that is highly visible under fluoroscopy. In another embodiment, the conductive material that the harness is formed from can be used to receive a band or coating of highly radiopaque material in order to enhance the visibility of the cardiac harness under fluoroscopy.

The present invention is directed to a cardiac harness system for treating the heart. In preferred embodiment, the cardiac harness is formed from a nitinol alloy wire and formed into rows of undulating or zig zag spring elements that provide an elastic force on the heart. The rows of spring elements are connected together by grip pads typically formed of a polymer material. Generally, under standard fluoroscopy, the nitinol alloy is visible, however, it is preferred that the radiopacity be increased to assist in delivering and implanting the cardiac harness onto the heart. Accordingly, in one embodiment of the invention, the cardiac harness is coated with a polymer, such as a silicone rubber, that is impregnated with a radiopaque material, such as barium sulfate (BaSO₄). The silicone rubber coating is loaded with a percentage of barium sulfate which is highly radiopaque and more visible under fluoroscopy than is the cardiac harness without the barium-sulfate-loaded silicone rubber coating.

It is preferred that the cardiac harness of the present invention be coupled with electrodes and a cardiac rhythm management system for the purpose of providing a defibrillating shock or pacing/sensing therapy to a patient. Thus, the description herein and the drawings describe use of the cardiac harness in conjunction with electrodes and a cardiac rhythm management device. Alternatively, the cardiac harness can be formed without the electrodes and the cardiac rhythm management device. In the embodiment without the electrodes (not shown) the rows of spring elements are continuous in a circumferential direction around the heart, and are connected by grip pads as disclosed herein. Thus, for discussion herein, the cardiac harness will be described in conjunction with electrodes in a cardiac rhythm management device, but the invention relating to the higher radiopacity in the cardiac harness can be applied with or without the electrodes and the cardiac rhythm management device.

The cardiac harness system of the present invention couples a highly radiopaque cardiac harness for treating the heart coupled with a cardiac rhythm management device. More particularly, the cardiac harness includes rows or undulating strands of spring elements that provide a compressive force on the heart during diastole and systole in order to relieve wall stress pressure on the heart. Associated with the cardiac harness is a cardiac rhythm management device for treating any number of irregularities in heart beat due to, among other reasons, congestive heart failure. Thus, the cardiac rhythm management device associated with the cardiac harness can include one or more of the following: an implantable cardioverter/defibrillator with associated leads and electrodes; a cardiac pacemaker including leads and electrodes used for sensing cardiac function and providing pacing stimuli to treat synchrony of both vessels; and a combined implantable cardioverter/defibrillator and pacemaker, with associated leads and electrodes to provide a defibrillation shock and/or pacing/sensing functions.

The cardiac harness system includes various configurations of panels connected together to at least partially surround the heart and assist the heart during diastole and systole. The cardiac harness system also includes one or more leads having electrodes associated with the cardiac harness and a source of electrical energy supplied to the electrodes for delivering a defibrillating shock or pacing stimuli.

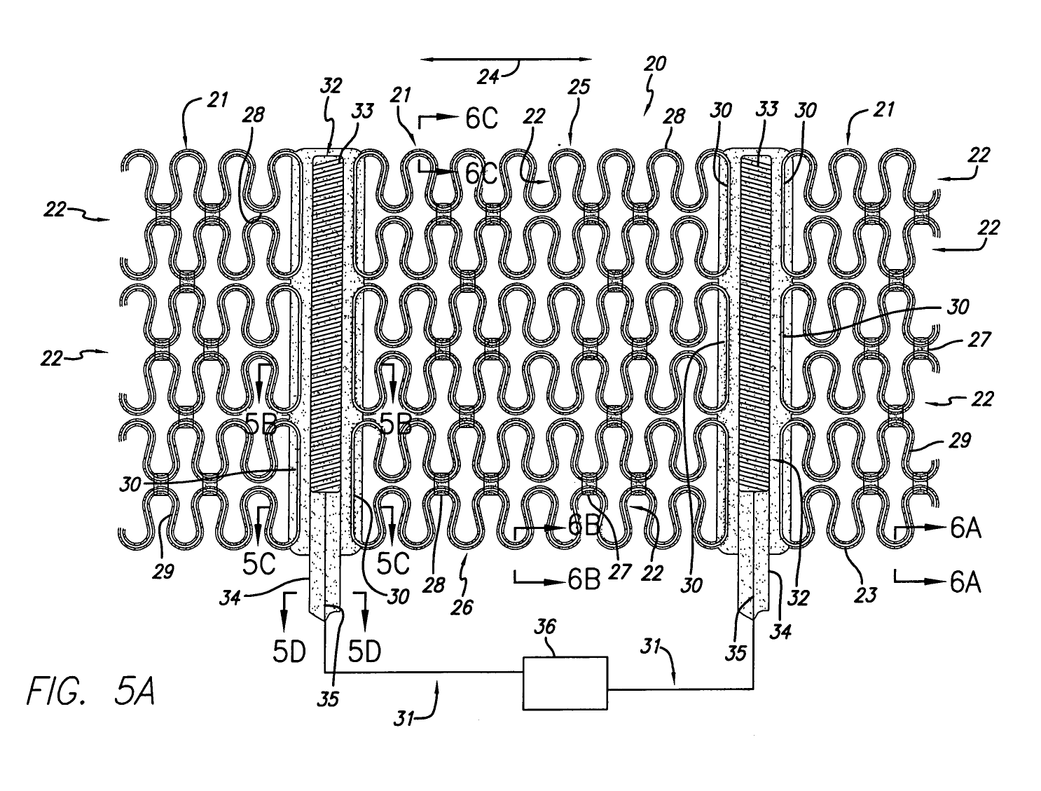

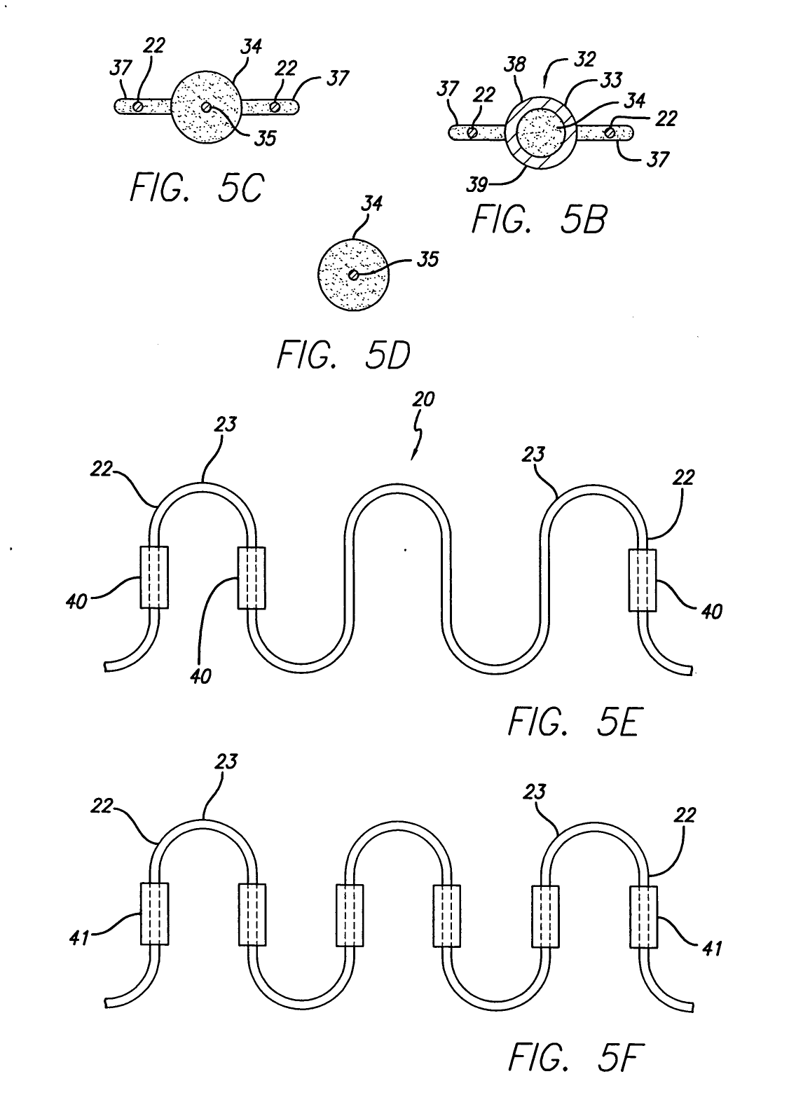

In one embodiment of the invention, as shown in a flattened configuration in FIG. 5A, a cardiac harness 20 includes two panels 21 of generally continuous undulating strands 22. A panel includes rows or undulating strands of hinges or spring elements that are connected together and that are positioned between a pair of electrodes, the rows or undulations being highly elastic in the circumferential direction and, to a lesser extent, in the longitudinal direction. In this embodiment, the undulating strands have U-shaped hinges or spring elements 23 capable of expanding and contracting circumferentially along directional line 24. The cardiac harness has a base or upper end 25 and an apex or lower end 26. The undulating strands are highly elastic in the circumferential direction when placed around the heart 10, and to a lesser degree in a direction parallel to the longitudinal axis 15 of the heart. Similar hinges or spring elements are disclosed in co-pending and co-assigned U.S. Ser. No. 10/656,722 filed Sep. 5, 2003, the entire contents of which are incorporated herein by reference. While the FIG. 5A embodiment appears flat for ease of reference, in use it is substantially cylindrical (or tapered) to conform to the heart and the right and left side panels would actually be one panel and there would be no discontinuity in the undulating strands.

The undulating strands 22 provide a compressive force on the epicardial surface of the heart thereby relieving wall stress. In particular, the spring elements 23 expand and contract circumferentially as the heart expands and contracts during the diastolic and systolic functions. As the heart expands, the spring elements expand and resist expansion as they continue to open and store expansion forces. During systole, as the heart 10 contracts, the spring elements will contract circumferentially by releasing the stored bending forces thereby assisting in both the diastolic and systolic function.

As just discussed, bending stresses are absorbed by the spring elements 23 during diastole and are stored in the elements as bending energy. During systole, when the heart pumps, the heart muscles contract and the heart becomes smaller. Simultaneously, bending energy stored within the spring elements 23 is at least partially released, thereby providing an assist to the heart during systole. In a preferred embodiment, the compressive force exerted on the heart by the spring elements of the harness comprises about 10% to 15% of the mechanical work done as the heart contracts during systole. Although the harness is not intended to replace ventricular pumping, the harness does substantially assist the heart during systole.

The undulating strands 22 can have varying numbers of spring element 23 depending upon the amplitude and pitch of the spring elements. For example, by varying the amplitude of the pitch of the spring elements, the number of undulations per panel will vary as well. It may be desired to increase the amount of compressive force the cardiac harness 20 imparts on the epicardial surface of the heart, therefore the present invention provides for panels that have spring elements with lower amplitudes and a shorter pitch, thereby increasing the expansion force imparted by the spring element. In other words, all other factors being constant, a spring element having a relatively lower amplitude will be more rigid and resist opening, thereby storing more bending forces during diastole. Further, if the pitch is smaller, there will be more spring elements per unit of length along the undulating strand, thereby increasing the overall bending force stored during diastole, and released during systole. Other factors that will affect the compressive force imparted by the cardiac harness onto the epicardial surface of the heart include the shape of the spring elements, the diameter and shape of the wire forming the undulating strands, and the material comprising the strands.

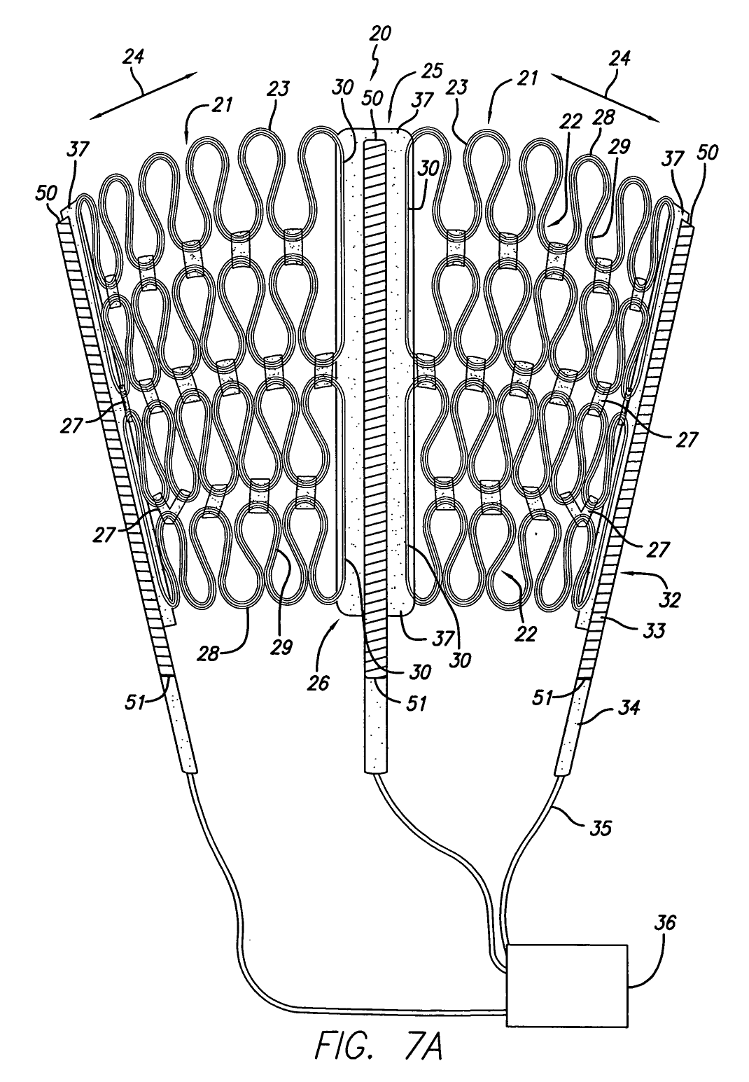

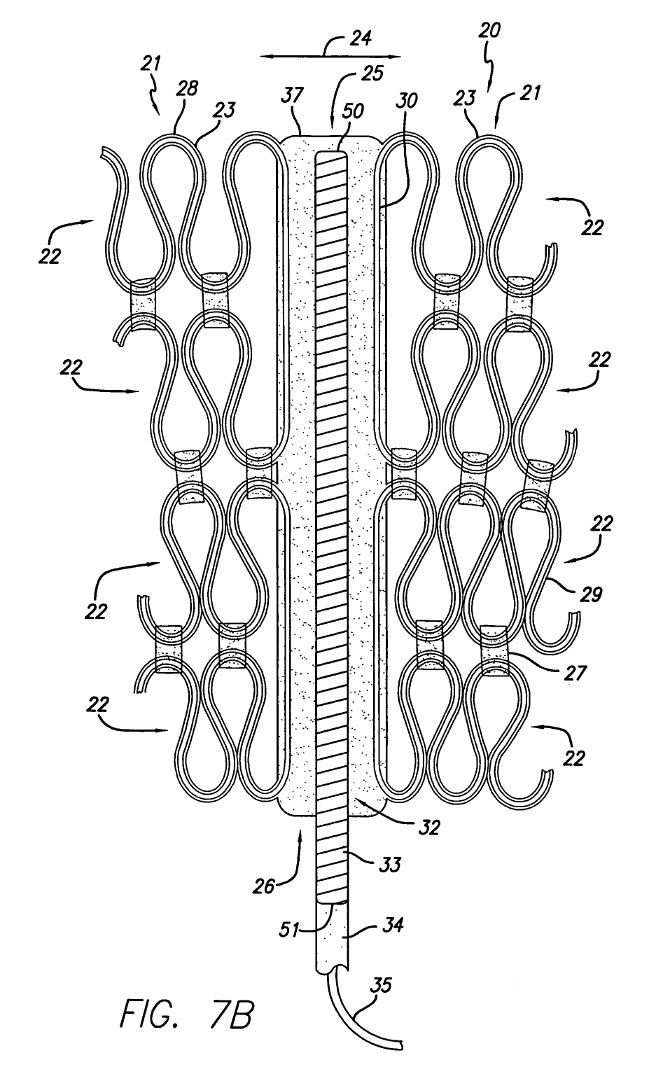

As shown in FIG. 5A, the undulating strands 22 are connected to each other by grip pads 27. In the embodiments shown in FIG. 5A, adjacent undulating strands are connected by one or more grip pads attached at the apex 28 of the spring elements 23. The number of grip pads between adjacent undulating strands is a matter of choice and can range from one grip pad between adjacent undulating strands, to one grip pad for every apex on the undulating strand. Importantly, the grip pads should be positioned in order to maintain flexibility of the cardiac harness 20 without sacrificing the objectives of maintaining the spacing between adjacent undulating strands to prevent overlap and to enhance the frictional engagement between the grip pads and the epicardial surface of the heart. Further, while it is desirable to have the grip pads attached at the apex of the spring elements, the invention is not so limited. The grip pads 27 can be attached anywhere along the length of the spring elements, including the sides 29. Further, the shape of the grip pads 27, as shown in FIG. 5A, can vary to suit a particular purpose. For example, grip pad 27 can be attached to the apex 28 of one undulating strand 22, and be attached to two apices on an adjacent undulating strand (see FIG. 7A). As shown in FIG. 5A, all of the apices point toward each other, and are said to be “out-of-phase.” If the apices of the undulations were aligned, they would be “in-phase.” The apices are all out-of-phase since the number of spring elements in each undulating strand is the same, however, the invention contemplates that the number of spring elements in each undulating strand may vary since the heart is tapered from its base near the top to its apex 13 at the bottom. Thus, there would be more spring elements and a longer undulating strand per panel at the top or base of the cardiac harness than at the bottom of the cardiac harness near the apex of the heart. Accordingly, the cardiac harness would be tapered from the relatively wide base to a relatively narrow bottom toward the apex of the heart, and this would affect the alignment of the apices of the spring elements, and hence the ability of the grip pads 27 to align perfectly and attach to adjacent apices of the spring elements. A further disclosure and embodiments relating to the undulating strands and the attachment means in the form of grip pads is found in co-pending and co-assigned U.S. Ser. No. 10/888,806 filed Jul. 8, 2004, the entire contents of which are incorporated herein by reference. While the connections between adjacent undulating strands 22 is preferably grip pads 27, in an alternative embodiment (not shown) the undulating strands are connected by interconnecting elements made of the same material as the strands. The interconnecting elements can be straight or curved as shown in FIGS. 8A-8B of commonly owned U.S. Pat. No. 6,612,979 B2, the entire contents of which is incorporated by reference herein.

It is preferred that the undulating strands 22 be continuous as shown in FIG. 5A. For example, every pair of adjacent undulating strands are connected by bar arm 30. It is preferred that the bar arms form part of a continuous wire that is bent to form the undulating strands, and then welded at its ends along the bar arm. The weld is not shown in FIG. 5A, but can be by any conventional method such as laser welding, fusion bonding, or conventional welding. The type of wire used to form the undulating strands may have a bearing on the method of attaching the ends of the wire used to form the undulating strand. For example, it is preferred that the undulating strands be made out of a nickel-titanium alloy, such as Nitinol, which may lose some of its superelastic or shape memory properties if exposed to high heat during conventional welding.

Associated with the cardiac harness of the present invention is a cardiac rhythm management device as previously disclosed. Thus, associated with the cardiac harness as shown in FIG. 5, are one or more electrodes for use in providing defibrillating shock. As can be seen immediately below, any number of factors associated with congestive heart failure can lead to fibrillation, acquiring immediate action to save the patient's life.

Diseased hearts often have several maladies. One malady that is not uncommon is irregularity in heartbeat caused by irregularities in the electrical stimulation system of the heart. For example, damage from a cardiac infarction can interrupt the electrical signal of the heart. In some instances, implantable devices, such as pacemakers, help to regulate cardiac rhythm and stimulate heart pumping. A problem with the heart's electrical system can sometimes cause the heart to fibrillate. During fibrillation, the heart does not beat normally, and sometimes does not pump adequately. A cardiac defibrillator can be used to restore the heart to normal beating. An external defibrillator typically includes a pair of electrode paddles applied to the patient's chest. The defibrillator generates an electric field between electrodes. An electric current passes through the patient's heart and stimulates the heart's electrical system to help restore the heart to regular pumping.

Sometimes a patient's heart begins fibrillating during heart surgery or other open-chest surgeries. In such instances, a special type of defibrillating device is used. An open-chest defibrillator includes special electrode paddles that are configured to be applied to the heart on opposite sides of the heart. A strong electric field is created between the paddles, and an electric current passes through the heart to defibrillate the heart and restore the heart to regular pumping.

In some patients that are especially vulnerable to fibrillation, an implantable heart defibrillation device may be used. Typically, an implantable heart defibrillation device includes an implantable cardioverter defibrillator (ICD) or a cardiac resynchronization therapy device (CRT-D) which usually has only one electrode positioned in the right ventricle, and the return electrode is the defibrillator housing itself, typically implanted in the pectoral region. Alternatively, an implantable device includes two or more electrodes mounted directly on, in or adjacent the heart wall. If the patient's heart begins fibrillating, these electrodes will generate an electric field therebetween in a manner similar to the other defibrillators discussed above.

Testing has indicated that when defibrillating electrodes are applied external to a heart that is surrounded by a device made of electrically conductive material, at least some of the electrical current disbursed by the electrodes is conducted around the heart by the conductive material, rather than through the heart. Thus, the efficacy of defibrillation is reduced. Accordingly, the present invention includes several cardiac harness embodiments that enable defibrillation of the heart and other embodiments disclose means for defibrillating, resynchronization, left ventricular pacing, right ventricular pacing, and biventricular pacing/sensing.

In further keeping with the invention, the cardiac harness 20 includes a pair of leads 31 having conductive electrode portions 32 that are spaced apart and which separate panels 21. As shown in FIG. 5A, the electrodes are formed of a conductive coil wire 33 that is wrapped around a non-conductive member 34, preferably in a helical manner. A conductive wire 35 is attached to the coil wire and to a power source 36. As used herein, the power source 36 can include any of the following, depending upon the particular application of the electrode: a pulse generator; an implantable cardioverter/defibrillator; a pacemaker; and an implantable cardioverter/defibrillator coupled with a pacemaker. In the embodiment shown in FIG. 5A, the electrodes are configured to deliver an electrical shock, via the conductive wire and the power source, to the epicardial surface of the heart so that the electrical shock passes through the myocardium. Even though the electrodes are spaced so that they would be about 180° apart around the circumference of the heart in the embodiment shown, they are not so limited. In other words, the electrodes can be spaced so that they are about 45° apart, 60° apart, 90° apart, 120° apart, or any arbitrary arc length spacing, or, for that matter, essentially any arc length apart around the circumference of the heart in order to deliver an appropriate electrical shock. As previously described, it may become necessary to defibrillate the heart and the electrodes 32 are configured to deliver an appropriate electrical shock to defibrillate the heart.

Still referring to FIG. 5A, the electrodes 32 are attached to the cardiac harness 20, and more particularly to the undulating strands 22, by a dielectric material 37. The dielectric material insulates the electrodes from the cardiac harness so that electrical current does not pass from the electrode to the harness thereby undesirably shunting current away from the heart for defibrillation. Preferably, the dielectric material covers the undulating strands 22 and covers at least a portion of the electrodes 32. In the FIG. 5A embodiment, the middle panel undulating strands are covered with the dielectric material while the right and left side panels are bare metal. While it is preferred that all of the undulating strands of the panels be coated with the dielectric material, thereby insulating the harness from the electric shock delivered by the electrodes, some or all of the undulating strands can be bare metal used to deliver the electrical shock to the epicardial surface of the heart for defibrillation or for pacing.

As will be described in more detail, the electrodes 32 have a conductive discharge first surface 38 that is intended to be proximate to or in direct contact with the epicardial surface of the heart, and a conductive discharge second surface 39 that is opposite to the first surface and faces away from the heart surface. As used herein, the term “proximate” is intended to mean that the electrode is positioned near or in direct contact with the outer surface of the heart, such as the epicardial surface of the heart. The first surface and second surface typically will not be covered with the dielectric material 37 so that the bare metal conductive coil can transmit the electrical current from the power source (pulse generator), such as an implantable cardioverter/defibrillator (ICD or CRT-D) 36, to the epicardial surface of the heart. In an alternative embodiment, either the first or the second surface may be covered with dielectric material in order to preferentially direct the current through only one surface. Further details of the construction and use of the leads 31 and electrodes 33 of the present invention, in conjunction with the cardiac harness, will be described more fully herein.

Importantly, the dielectric material 37 used to attach the electrodes 32 to the undulating strands 22 insulates the undulating strands from any electrical current discharged through the conductive metal coils 33 of the electrodes. Further, the dielectric material in this embodiment is flexible so that the electrodes can serve as a seam or hinge to fold the cardiac harness 20 into a lower profile for minimally invasive delivery. Thus, as will be described in more detail, the cardiac harness can be folded along its length, along the length of the electrodes, in order to reduce the profile for intercostal delivery, for example through the rib cage or other area typically used for minimally invasive surgery for accessing the heart. Minimally invasive approaches involving the heart typically are made through subxiphoid, subcostal or intercostal incisions. When the cardiac harness is folded, it can be reduced into a circular or a more or less oval shape, both of which promote minimally invasive procedures.

In further keeping with the invention, cross sectional views of the leads 31 and the electrode portion 32 are shown in FIGS. 5B, 5C, and 5D. As shown in FIG. 5B, the electrode 32 has the coil wire 33 wrapped around the non-conducting member 34 in a helical pattern. The dielectric material 37 provides a spaced connection between the electrode and the bar arms 30 at the ends of the undulating strands 22. The electrodes do not touch or overlap with the bar arms or any portion of the undulating strands. Instead, the dielectric material provides the attachment means between the electrodes and the bar arms of the undulating strands. Thus, the dielectric material 37 not only acts as an insulating non-conductive material, but also provides attachment means between the undulating strands and the electrodes. Because the dielectric material 37 is relatively thin at the attachment points, it is highly flexible and permits the electrodes to be flexible along with the cardiac harness panels 21, which will expand and contract as the heart beats as previously described.

Referring to FIGS. 5B, 5C and 5D, the non-conductive member 34 preferably is made from the same material as the dielectric material 37, typically a silicone rubber or similar material. While it is preferred that the dielectric material be made from silicone rubber, or a similar material, it also can be made from Parylene™ (Union Carbide), polyurethanes, elastomers, PTFE, elastomers, TFE, and ePTFE. As can be seen, the non-conductive member provides support for the dielectric material to attach the bar arms 30 of the undulating strands 22 in order to connect the strands to the electrode 32. In further keeping with the invention, the non-conductive member 34 and the dielectric material 37 each can be loaded with a radiopaque material such as barium sulfate (BaSO₄) or a similar material to improve the radiopacity of the cardiac harness. The BaSO₄ is loaded into the non-conductive member 34 and the dielectric material 37 by known means, and can be loaded in a range of about 5% up to about 35% by weight. Some or any portion of the non-conductive member 34 and the dielectric material 37 can be loaded with the BaSO₄. Further, while BaSO₄ is preferred, other radiopaque materials can be used such as powders of bismuth subcarbonate, bismuth trioxide, bismuth oxychloride, tungsten, tantalum, iridium, gold, or other dense metals having a high molecular weight. Importantly, any material used for loading the non-conductive member 34 and dielectric material 37 should be biocompatible.

Alternatively, to improve the radiopacity of the cardiac harness 20, metal bands 40 are attached to the undulating strands 22, as shown in FIG. 5E. The metal bands are a high density or high molecular mass material such as gold, platinum, platinum iridium, silver, tungsten, and the like. Preferably, the bands are crimped onto the undulating strands before a dielectric material or other insulating material is applied to the undulating strands 22. Further, it is preferred that radiopaque bands 40 be crimped or coated onto undulating strands 22, and at preferably straight portions on the undulating strands so that the expansion of the hinge joints in the undulating strands is not compromised. The radiopaque bands 40 are highly visible under fluoroscopy, but not affect the stretch or fatigue properties of the undulating strands, typically formed from nitinol wire. The radiopaque bands 40 can have a split and the undulating strand 22 pushed through the slit so that the bands can then be crimped or swaged or bonded onto the wire of the undulating strand prior to jacketing or covering the undulating strand with a silicone tubing or other form of dielectric material. The number and placement of radiopaque bands 40 is a matter of choice. For example, the undulating strands 22, at the top and bottom (base and apex) of the cardiac harness 20 can be fitted with a number of radiopaque bands to define the extremities of the cardiac harness. Further, a middle row of undulating strands 22 can be fitted with radiopaque bands 40 to assist in visualizing the middle part of the cardiac harness under fluoroscopy. Further, it may be advantageous to measure the distance between two radiopaque bands 40 as the heart expands and contracts in order to evaluate the amount of stretch of one of the spring elements 23. Measuring the amount of stretch of spring elements 23 by calculating the movement of radiopaque markers 40 can assist the physician in determining if the heart is reverse remodeling over a period of time with the assistance of the cardiac harness 20.

Alternatively, the radiopacity of cardiac harness 20 can be increased by coating all of or a portion of undulating strands 22 of the cardiac harness 20 with a high density or high molecular mass material. For example, as shown in FIG. 5F, a portion of an undulating strand 22 has been coated with a high density or high molecular mass metal such as platinum, platinum iridium, gold, silver, tungsten, or the like. The coating 41 can be applied in any known manner such as by sputtering, plasma deposition, or similar methods known in the art. Preferably, the radiopaque coating 41 is placed only on the straight portions of the undulating strands 22 so that the expansion characteristics of the spring elements 23 are not compromised. Typically, radiopaque coating 41 will be only 5 to 20 angstroms thick, so that if a portion of the spring elements 23 are covered by the coating, it should not interfere with the elastic properties of the spring elements 23. The radiopaque coating 41 can be applied to any of the undulating strands 23, or any portion of the undulating strands as desired. For example, the base and apex rows of undulating strands can be coated with radiopaque coating 41, or any portion can be coated. Further, a dielectric material as previously described, can be formed or coated over the undulating strands and the radiopaque coating 41 in order to insulate the undulating strands, preferably made from nitinol wire, from any electrical charges from an internal or external defibrillator.

Alternatively, grip pads 27, used to connect the undulating strands 22 of the cardiac harness 20 also are formed of a polymer material as herein described. The grip pads 27 also can be loaded with a highly radiopaque material such as BaSO₄, or the other material previously described to enhance the radiopacity of the cardiac harness. Thus, some or all of the grip pads 27 can be loaded with BaSO₄ to enhance the visibility of the cardiac harness under fluoroscopy.

A conductive wire 35 extends through the non-conducting member and attaches to the proximal end of the coil wire 33 so that when an electrical current is delivered from the power source 36 through conductive wire 35, the electrode coil 33 will be energized. The conductive wire 35 is also covered by non-conducting material 34. Referring to FIG. 5D, it can be seen that the non-conductive member 34 continues to extend beyond the bottom (apex) of the cardiac harness and that conductive wire 35 continues to extend out of the non-conductive member and into the power source 36. In the embodiment shown in FIGS. 5B-5D, the cardiac harness is insulated from the electrodes by the dielectric material 37 so that there is no shunting of electrical currents by the cardiac harness 20 from the electrical shock delivered by the electrodes during defibrillation or pacing functions.

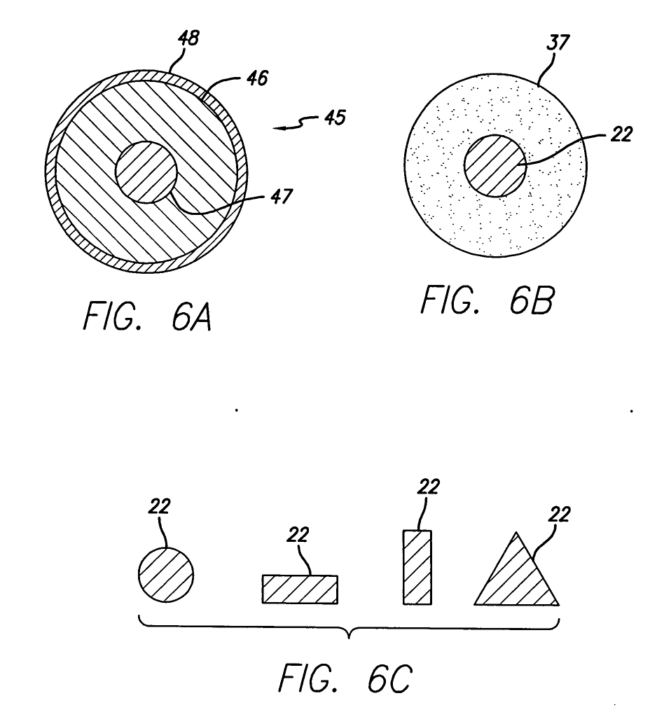

While it is preferred that the cardiac harness 20 be comprised of undulating strands 22 made from a solid wire member, such as a superelastic or shape memory material such as Nitinol, and be insulated from the electrodes 32, it is possible to use some or all of the undulating strands to deliver the electrical shock to the epicardial surface of the heart. For example, as shown in FIG. 6A, a composite wire 45 can be used to form the undulating strands 22 and, importantly, to effectively transmit current to deliver an electrical shock to the epicardial surface of the heart. The composite wire 45 includes a current conducting wire 47 made from, for example silver (Ag), and which is covered by a Nitinol tube 46. In order to improve the surface conductivity of the outer Nitinol tube 46, a highly conductive coating is placed on the Nitinol tube. For example, the Nitinol tube can be covered with a deposition layer of platinum (Pt) or platinum-iridium (Pt—Ir), or an equivalent material including iridium oxide (IROX). The composite wire, so constructed, will have superior mechanical performance to expand and contract due to the Nitinol tubing, and also will have improved electrical properties resulting from the current conducting wire 47 and improved electrolytic/electrochemical properties via the surface layer of platinum-iridium. Thus, if some portion or all of the undulating strands 22 are made from a composite wire 45, the cardiac harness 20 will be capable of delivering a defibrillating shock on selected portions of the heart via the undulating strands and will also function to impart compressive forces as previously described. Further, the platinum, platinum-iridium, and iridium oxide are highly radiopaque and are highly visible under fluoroscopy as previously described.

In contrast to the current conducting undulating strands of FIG. 6A, are the non-conducting insulated undulating strands 22 as shown by cross sectional view FIG. 6B. As previously described, some or all of the undulating strands 22 can be covered with dielectric material 37 in order to insulate the strands from the electrical current delivered through the electrodes while delivering shock on the epicardial surface of the heart. Thus, as shown in FIG. 6B, the undulating strands 22 are covered by dielectric material 37 to provide insulation from the electrical shock delivered by the electrodes 32, yet maintain the flexibility and the expansive properties of the undulating strands.

An important aspect of the invention is to provide a cardiac harness 20 that can be implanted minimally invasively and be attached to the epicardial surface of the heart, without requiring sutures, clips, screws, glue or other attachment means. Importantly, the undulating strands 22 may provide relatively high frictional engagement with the epicardial surface, depending on the cross-sectional shape of the strands. For example, in the embodiment disclosed in FIG. 6C, the cross-sectional shape of the undulating strands 22 can be circular, rectangular, triangular or for that matter, any shape that increases the frictional engagement between the undulating strands and the epicardial surface of the heart. As shown in FIG. 6C, the middle cross-section view having a flat rectangular surface (wider than tall) not only has a low profile for enhancing minimally invasive delivery of the cardiac harness, but it also has rectangular edges that may have a tendency to engage and dig into the epicardium to increase the frictional engagement with the epicardial surface of the heart. With the proper cross-sectional shape for the undulating strands, coupled with the grip pads 27 having a high frictional engagement feature, the necessity for suturing, clipping, or further attachment means to attach the cardiac harness to the epicardial surface of the heart becomes unnecessary.

In another embodiment as shown in FIGS. 7A and 7B, a different configuration for cardiac harness 20 and the electrodes 32 are shown, as compared to the FIG. 5 embodiments. In FIGS. 7A and 7B, three electrodes are shown separating the three panels 21 with undulating strands 22 extending between the electrodes. As with previous embodiments, springs 23 are formed by the undulating strands so that the undulating strands can expand and contract during the diastolic and systolic functions, and apply a compressive force during both functions. The far side panel of FIG. 7A is not shown for clarity purposes. The position of the electrodes around the circumference of the heart is a matter of choice, and in the embodiment of FIG. 7A, the electrodes can be spaced an equal distance apart at about 120°. Alternatively, it may be important to deliver the electrical shock more through the right ventricle requiring the positioning of the electrodes closer to the right ventricle than to the left ventricle. Similarly, it may be more important to deliver an electrical shock to the left ventricle as opposed to the right ventricle. Thus, positioning of electrodes, as with other embodiments, is a matter of choice.

Still referring to FIGS. 7A and 7B, in this embodiment electrodes 32 extend beyond the bottom or apex portion of the cardiac harness 20 in order to insure that the electrical shock delivered by the electrodes is delivered to the epicardial surface of the heart and including the lower portion of the heart closer to the apex 13. Thus, the electrodes 22 have a distal end 50 and a proximal end 51 where the proximal end is positioned closer to the apex 13 of the heart and the distal end is positioned closer to the base or upper portion of the heart. As used herein, distal is intended to mean further into the body and away from the attending physician, and proximal is meant to be closer to the outside of the body and closer to the attending physician. The proximal ends of the electrodes are positioned closer to the apex of the heart and provide several functions, including the ability to deliver an electrical shock closer to the apex of the heart. The electrode proximal ends also function to provide support for the cardiac harness 20 and the panels 21, and lend support not only during delivery (as will be further described herein) but in separating the panels and in gripping the epicardial surface of the heart to retain the harness on the heart without slipping.

While the FIGS. 7A and 7B embodiments show electrodes 32 separating three panels 21 of the cardiac panel 20, more or fewer electrodes and panels can be provided to suit a particular application. For example, in one preferred embodiment, four electrodes 32 separate four panels 21, so that two of the electrodes can be positioned on opposite sides of the left ventricle and two of the electrodes can be positioned on opposite sides of the right ventricle. In this embodiment, preferably all four electrodes would be used, with a first set of two electrodes on opposite sides of the right ventricle acting as one (common) electrode and a second set of two electrodes on opposite sides of the left ventricle acting as the opposite (common) electrode. Alternatively, two of the electrodes can be activated while the other two electrodes act as dummy electrodes in that they would not be activated unless necessary.

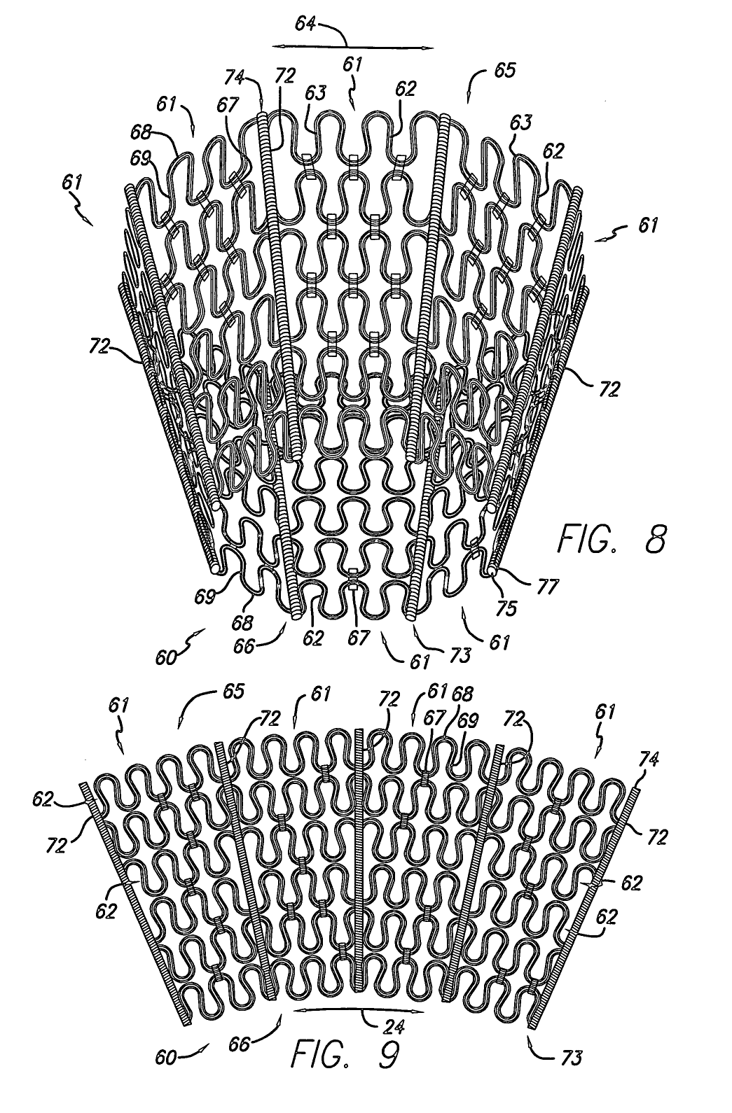

In another embodiment of the invention, as shown in FIGS. 8-11, cardiac harness 60 is similar to previously described cardiac harness 20. With respect to cardiac harness 60, it also includes panels 61 consisting of undulating strands 62. In the disclosed embodiments, the undulating strands are continuous and extend through coils as will be described. The undulating strands act as spring elements 63 as with prior embodiments that will expand and contract along directional line 64. The cardiac harness 60 includes a base or upper end 65 and an apex or lower end 66. In order to add stability to the cardiac harness 60, and to assist in maintaining the spacing between the undulating strands 62, grip pads 67 are connected to adjacent strands, preferably at the apex 68 of the springs. Alternatively, the grip pads 67 could be attached from the apex of one spring element to the side 69 of a spring element, or the grip pad could be attached from the side of one spring to the side of an adjacent spring on an adjacent undulating strand. In further keeping with the invention as shown in the FIGS. 8-11, in order to add stability and some mechanical stiffness to the cardiac harness 60, coils 62 are interwoven with the undulating strands, which together define the panels 61. The coils typically are formed of a coil of wire such as Nitinol or similar material (stainless steel, MP35N), and are highly flexible along their longitudinal length. The coils 72 have a coil apex 73 and a coil base 74 to coincide with the harness base 65 and the harness apex 66. The coils can be injected with a non-conducting material so that the undulating strands extend through gaps in the coils and through the non-conducting material. The non-conducting material also fills in the gaps which will prevent the undulating strands from touching the coils so there is no metal-to-metal touching between the undulating strands and the coils. Preferably, the non-conducting material is a dielectric material 77 that is formed of silicone rubber or equivalent material as previously described. Further, a dielectric material 78 also covers the undulating strands in the event a defibrillating shock or pacing stimuli is delivered to the heart via an external defibrillator (e.g., transthoracic) or other means.

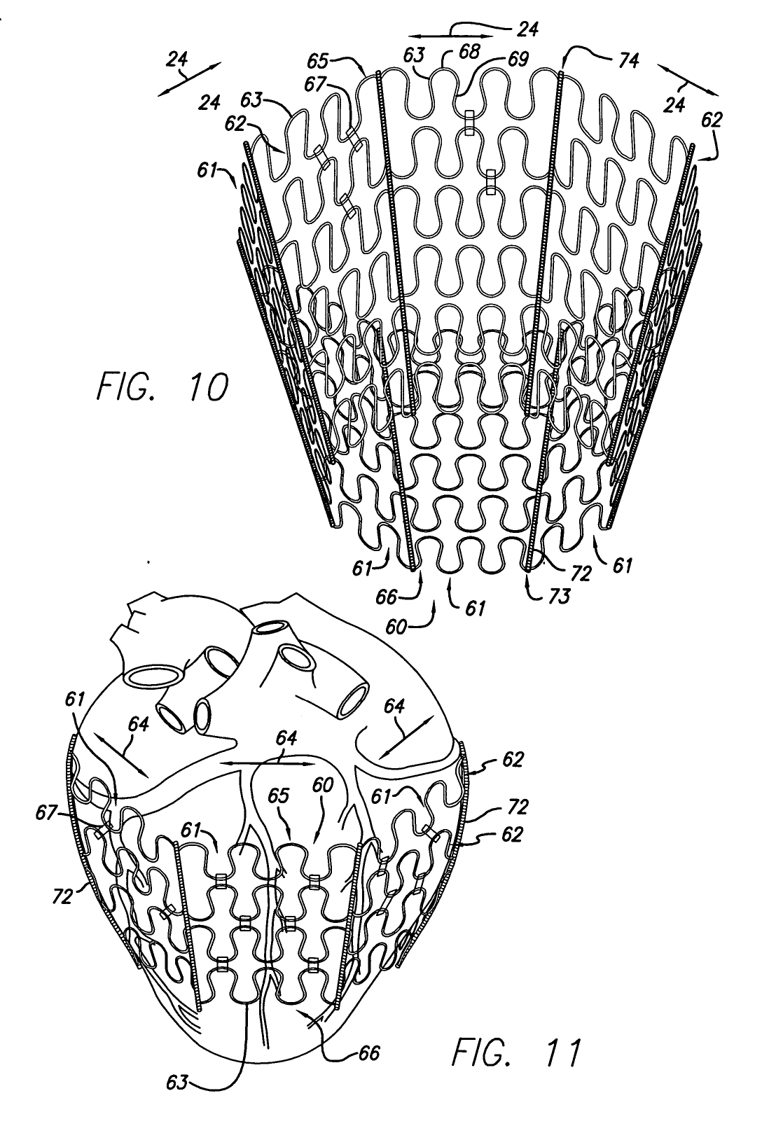

Importantly, coils 72 not only perform the function of being highly flexible and provide the attachment means between the coils and the undulating strands, but they also provide structural columns or spines that assist in deploying the harness 60 over the epicardial surface of the heart. Thus, as shown for example in FIG. 11, the cardiac harness 60 has been positioned over the heart and delivered by minimally invasive means, as will be described more fully herein. The coils 72, although highly flexible along their longitudinal length, have sufficient column strength in order to push on the apex 73 of the coils so that the base portion 74 of the coils and of the harness 65 slide over the apex of the heart and along the epicardial surface of the heart until the cardiac harness 60 is positioned over the heart, substantially as shown in FIG. 11.

Referring to the embodiments shown in FIGS. 8 and 10, the cardiac harness 60 has multiple panels 61 and multiple coils 72. More or fewer panels and coils can be used in order to achieve a desired result. For example, eight coils are shown in FIGS. 8 and 10, while fewer coils may provide a harness with greater flexibility since the undulating strands 62 would be longer in the space between each coil. Further, the diameter of the coils can be varied in order to increase or decrease flexibility and/or column strength in order to assist in the delivery of the harness over the heart. The coils preferably have a round cross-sectional wire in the form of a tightly wound spiral or helix so that the cross-section of the coil is circular. However, the cross-sectional shape of the coil need not be circular, but may be more advantageous if it were oval, rectangular, or another shape. Thus, if coils 72 had an oval shape, where the longer axis of the oval was parallel to the circumference of the heart, the coil would flex along its longitudinal axis and still provide substantial column strength to assist in delivery of the cardiac harness 60. Further, an oval-shaped coil would provide a lower profile for minimally invasive delivery. The wire cross-section also need not be round/circular, but can consist of a flat ribbon having a rectangular shape for low profile delivery. The coils also can have different shapes, for example they can be closed coils, open coils, laser-cut coils, wire-wound coils, multi-filar coils, or the coil strands themselves can be coiled (i.e., coiled coils). The electrode need not have a coil of wire, rather the electrode could be formed by a zig-zag-shaped wire (not shown) extending along the electrode. Such a design would be highly flexible and fatigue resistant yet still be capable of providing a defibrillating shock.

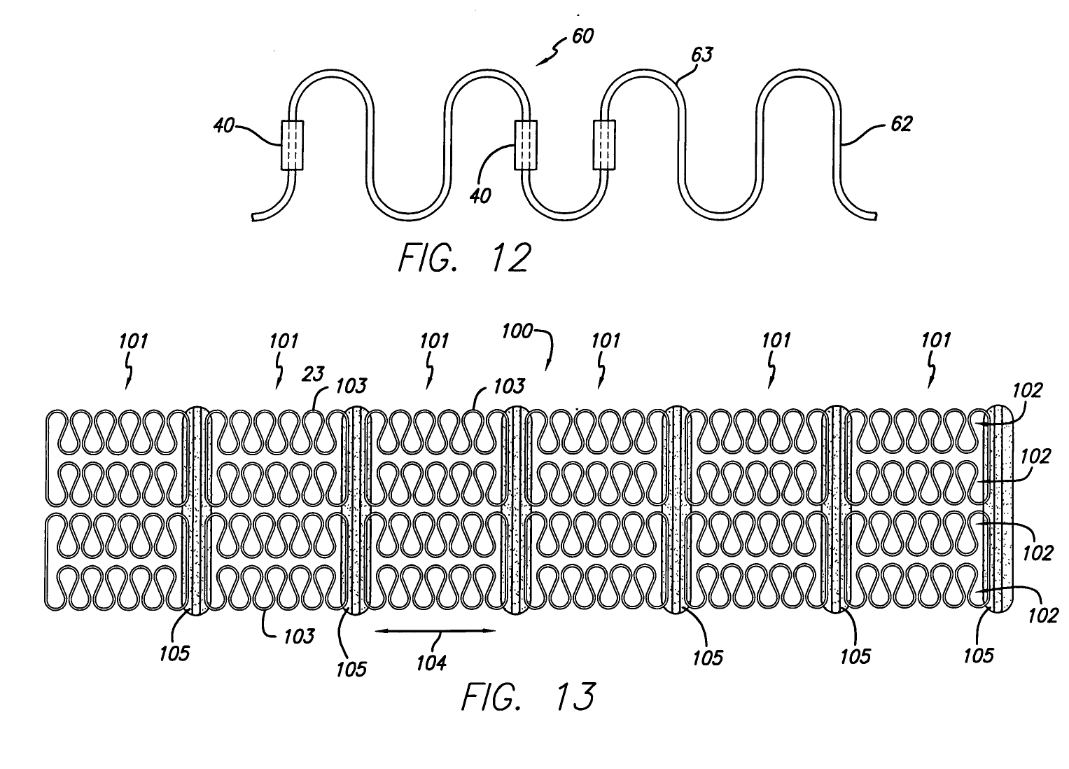

In one embodiment of the invention, shown in FIG. 12, a portion of a cardiac harness 60 includes a portion of undulating strands 62 that has enhanced radiopacity. More specifically, at least a portion of undulating strands 62 has a radiopaque band 40 formed of a high density or high molecular mass metal as previously described. The radiopaque band 40 is highly visible under fluoroscopy or other visualization means.

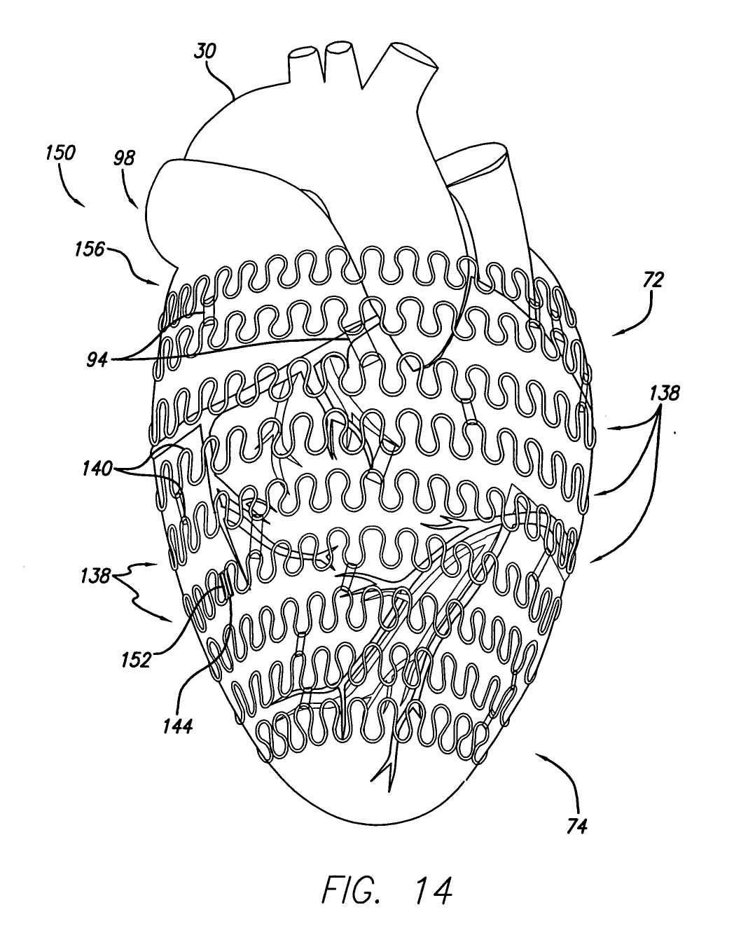

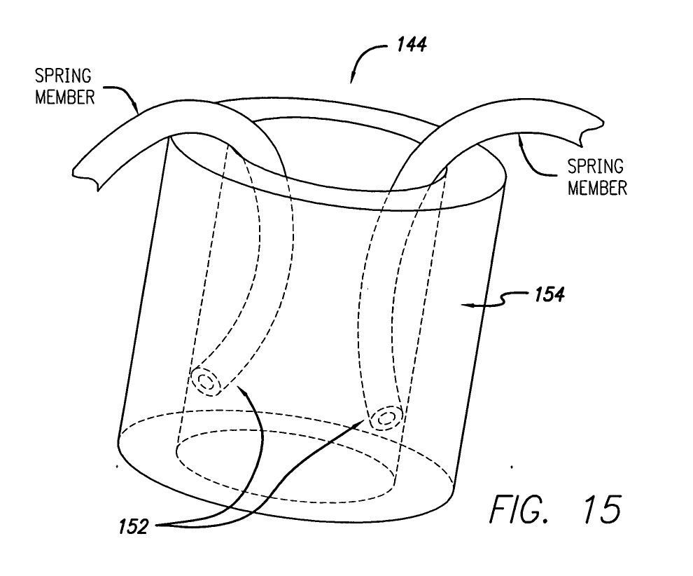

In another embodiment of the invention, shown in FIG. 13, cardiac harness 100 includes multiple panels 101 similar to those previously described. Further, undulating strands 102 form the panels and have multiple spring elements 103 that expand and contract along directional line 104, also as previously described for other embodiments. In the cardiac harness 100 shown in FIG. 13, the amplitude of the spring elements is relatively smaller than in other embodiments, and the pitch is higher, meaning there are more spring elements per unit of length relative to other embodiments. Thus, the cardiac harness 100 should generate higher bending forces as the heart expands and contracts during the diastolic and systolic cycles. In other words, the spring elements 103 of cardiac harness 100 will resist expansion, thereby imparting higher compressive forces on the wall of the heart during the diastolic function and will release these higher bending forces during the systolic function as the heart contracts. It may be important to provide undulating strands 102 that alternate in amplitude and pitch within a panel, starting at the base of the harness and extending toward the apex. For example, the pitch and amplitude of an undulating strand closer to the base or the harness may be configured to impart higher compressive forces on the epicardial surface of the heart than the undulating strands closer to the apex or the lower part of the harness. It also may be desirable to alternate the amplitude and pitch of the spring elements from one undulating strand to the next. Further, where multiple panels are provided, it may be advantageous to provide one amplitude and pitch of the spring elements of the undulating strands of one panel, and a different amplitude and pitch of the spring elements of the undulating strands of an adjacent panel. The FIG. 13 embodiment can be configured with electrodes as previously described in other embodiments, or with coils, both of which assist with the delivery of the cardiac harness by providing column support to the harness. The FIG. 13 panels 101 can be connected to each other by longitudinally extending connectors 105. The connectors 105 can be formed from any of the polymers disclosed herein (e.g., silicone rubber) and loaded with a radiopaque material (e.g., BaSO₄) to enhance the radiopacity of the panels.

It is to be understood that various materials and methods can be used to coat the harness with dielectric material. For example, in one embodiment, an etched harness is coated with a layer of Parylene™, which is a dielectric polymer available from Union Carbide. Other acceptable materials include silicone rubbers and urethanes, as well as various polymers and the like. The materials can be applied to an etched harness by various methods, such as dip coating and spraying.

In accordance with one embodiment, a cardiac harness preferably is formed into a desired shape before being coated with dielectric material. For example, in one embodiment, Nitinol wire preferably is first treated and shaped to develop a shape memory of a desired spring member structure. Silicone tubing is then pulled over the wire. The wire then is returned to its shape memory shape. In another embodiment, Nitinol wire is dip coated with an insulating material.

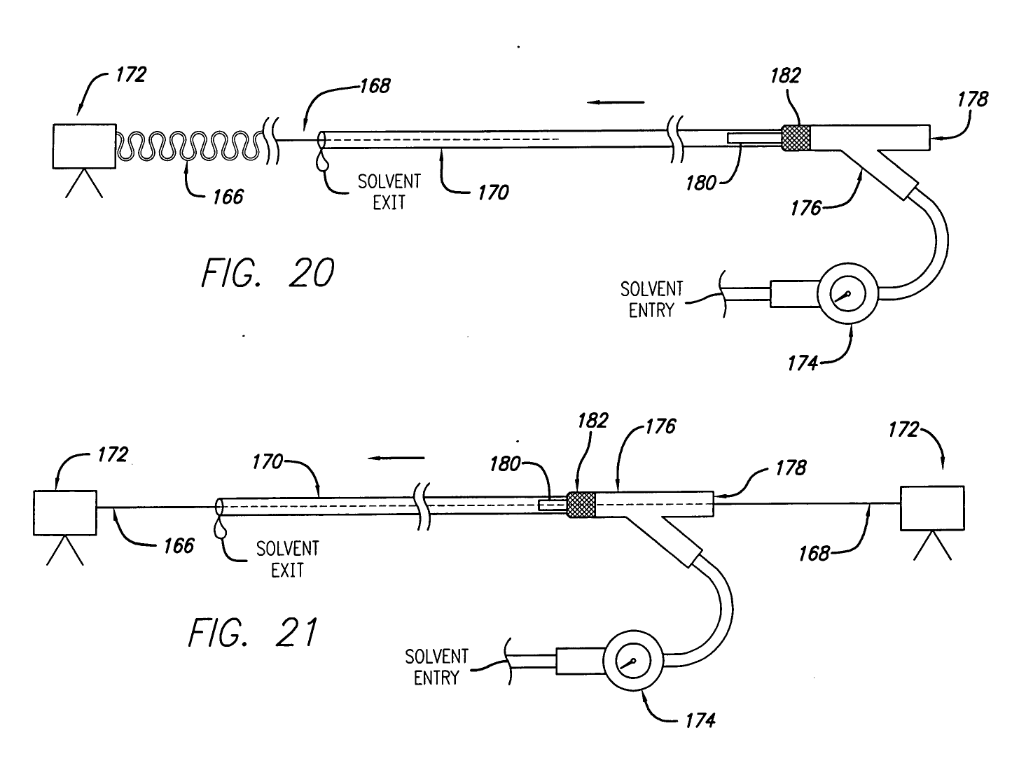

In another embodiment, a harness is electrically insulated by stretching an extruded tube of flexible dielectric material over the harness. In a further embodiment, another flexible dielectric tube is disposed on the opposite side of the harness to effectively sandwich the harness between layers of flexible expandable dielectric material. Gaps may be formed through the dielectric material to help communicate the electric field through the harness to the heart. Specific methods for insulating the wire are discussed in more detail below with reference to FIGS. 20 and 21

With next reference to FIG. 14, another embodiment of a cardiac harness 150 is illustrated disposed on a simulated heart 30. As shown, the cardiac harness is configured to circumferentially surround the heart and extend longitudinally from a base portion 72 to an apex portion 74 of the heart. The harness has a plurality of circumferentially extending rings 138 disposed longitudinally adjacent to one another. Each ring has a plurality of interconnected spring members 140 covered with a dielectric material. The spring members shown in FIG. 14 are similar to the spring members discussed above with reference to FIG. 5A. A plurality of nonconductive connectors 94 interconnects adjacent rings and can be formed of the same material as the grip pads 27 in FIG. 5A. In fact, in one embodiment, connectors 94 are the same as grip pads 27 in FIG. 5A. The nonconductive connectors 94 have a length oriented longitudinally relative to the rings so as to create space between adjacent rings. Preferably, the nonconductive connectors are formed of a semi-compliant design using silicone rubber or other similar material and can be loaded with a radiopaque material as previously described.

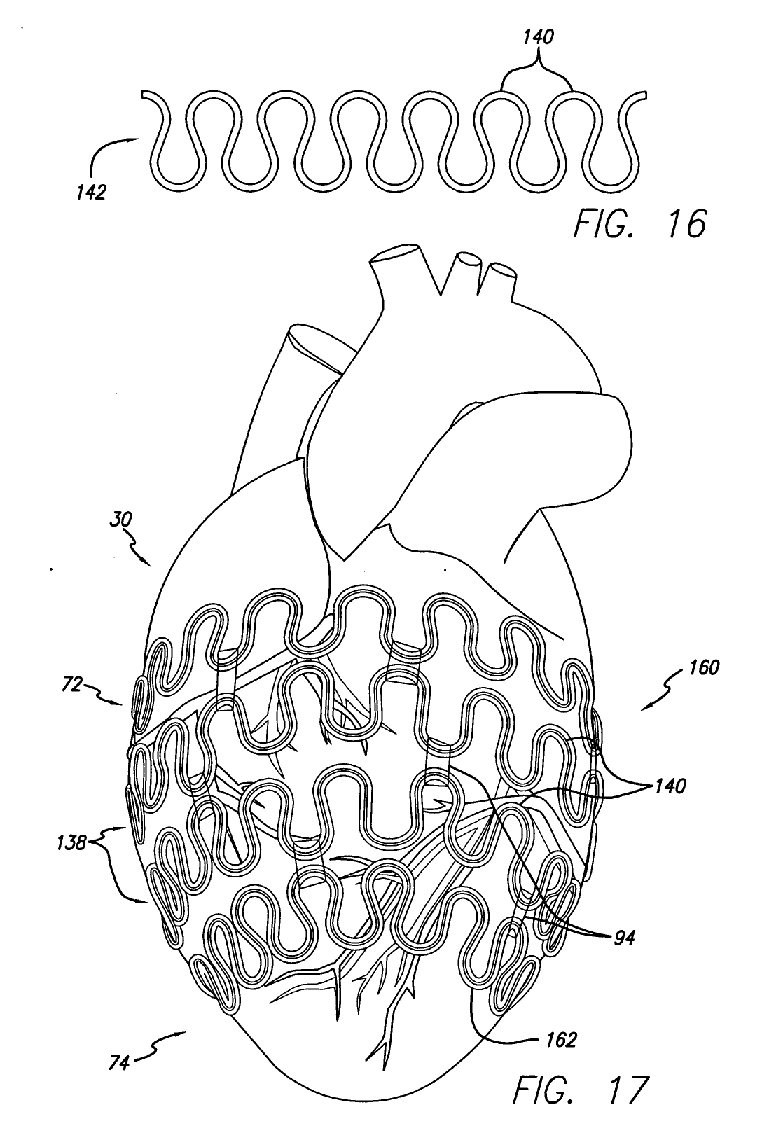

In one embodiment, each ring initially has an elongate strand or series of spring elements 142, as shown in FIG. 16. Each elongate strand has a series of the above-discussed spring members 140. During manufacturing of the cardiac harness, each elongate strand is cut to a length such that when opposite ends of the elongate strand are bonded together, the elongate strand assumes the ring-shaped configuration shown in FIG. 14. It will be appreciated that the lengths of the elongate strands are selected such that the resulting rings 138 are sized in conformity with the general anatomy of the patient's heart. More specifically, strands in the apex portion 74 of the harness are not as long as the strands used to form the base portion 72. As such, the harness generally tapers from the base toward the apex in order to generally follow the shape of the patient's heart. In another embodiment, the diameter of a ring at the base of the harness is smaller than the diameter of the adjacent ring. In this embodiment, the harness has a greatest diameter at a point between the base and apex ends, and tapers from that point to both the base and apex ends. Preferably, the point of greatest diameter is closer to the base end than to the apex end. It is contemplated that the lengths of the strands, as well as the sizes of the spring members, may be selected according to the intended size of the cardiac harness and/or the amount of compressive force the harness is intended to impart to the patient's heart.

As shown in FIG. 14, opposite ends 152 of each circumferentially extending ring 138 are attached to one another by a connective junction 144. In one embodiment, illustrated in FIG. 15, each connective junction has a small tube segment 154 into which the opposite ends of the ring are inserted. The tube segment serves to prevent the opposite ends of the ring from tearing loose from one another after the harness is placed on the heart. Preferably, each tube segment is filled with a dielectric material such as silicone, or other similar material after the ring-ends are placed therein.

With continued reference to FIG. 14, the right side 98 of the base portion 72 of the harness 150 has partial strands 156 of interconnected spring members 140. Preferably, the partial strands are connected to the adjacent full ring 138 in a manner so that the partial strands are stretched. As such, the partial strands will bend inwardly to “cup” the upper portion of the right atrium, as simulated in FIG. 14.

It will be appreciated that because the rings are coated with dielectric material and are interconnected by nonconductive material, each ring is electrically isolated from the other rings in the harness. As such, there is no electrical continuity either circumferentially about the harness or longitudinally along the harness. Thus, if an electric current created by a defibrillation device is applied to a patient who has a harness placed on their heart, the electric current passes through the heart rather than being conducted around the heart through the harness. As a result, the effectiveness of the defibrillation is not defeated by the presence of the harness.

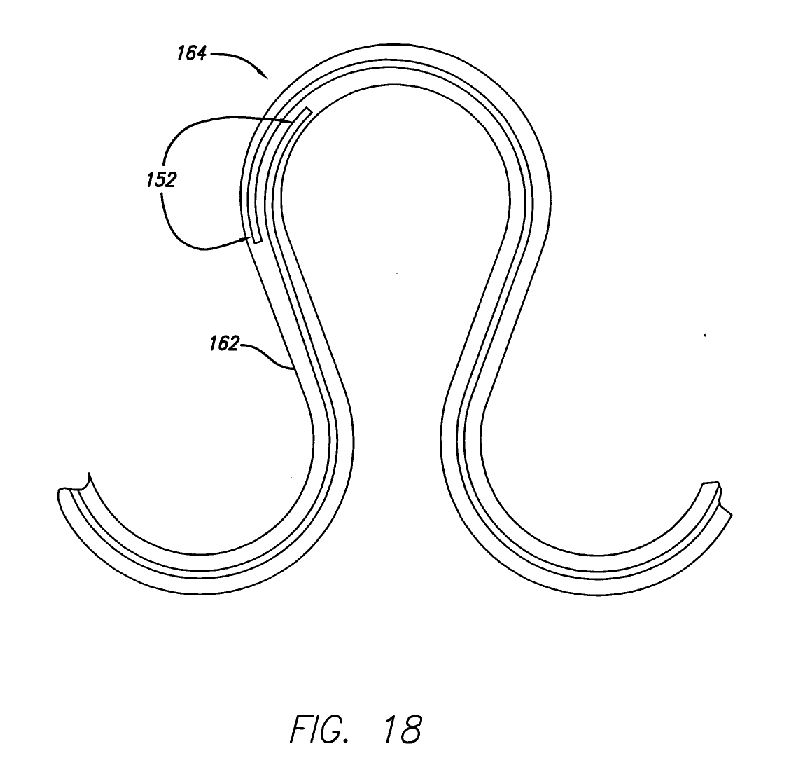

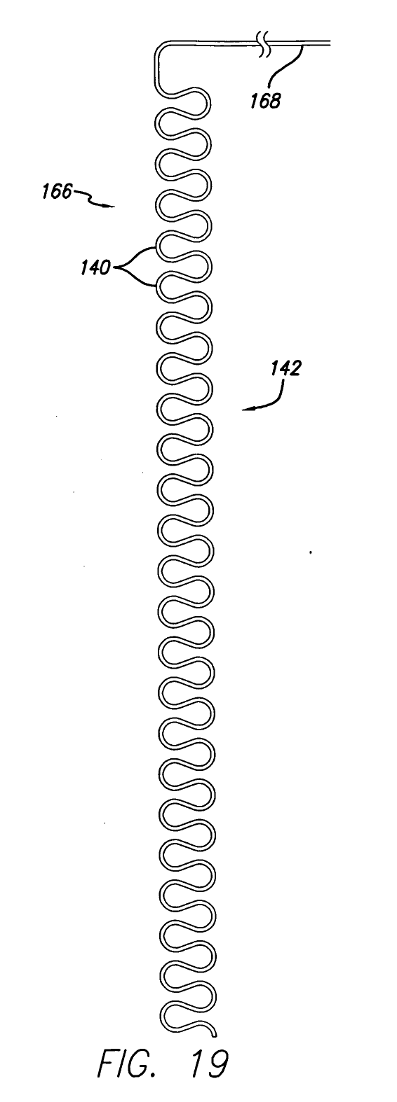

FIG. 17 illustrates another embodiment of a cardiac harness 160 disposed on a simulated heart 30. As shown, the cardiac harness is configured to circumferentially surround the heart and extends longitudinally from a base portion 72 to an apex portion 74 of the heart. The harness has a plurality of circumferentially extending rings 138. Each ring includes a plurality of interconnected spring members 140 covered with a dielectric material 162. The spring members shown in FIG. 17 are similar to the spring members discussed above with reference to FIGS. 2A and 2B. A plurality of nonconductive connectors 94 interconnects adjacent rings. In one embodiment, the nonconductive connectors are formed of the dielectric material which covers the spring members.

In one embodiment, each ring 138 initially has an elongate strand 142, as discussed above with reference to FIG. 16. Each elongate strand includes a series of the above-discussed spring members 140. During manufacturing of the cardiac harness, each elongate strand is cut to a length such that when opposite ends of the elongate strand are secured together, the elongate strand assumes the ring-shaped configuration shown in FIG. 17. The lengths of the elongate strands are selected such that the resulting rings are sized in conformity with the general anatomy of the patient's heart. In one embodiment, the lengths of the strands, as well as the sizes of the spring members, are selected according to the intended size of the cardiac harness and/or the amount of compressive force the harness is intended to impart to the patient's heart.

Once the elongate strands are formed into the ring-shaped configuration shown in FIG. 17, the rings 138 are covered with a dielectric material. The dielectric covering is configured to prevent an electric current applied by a defibrillation device from being communicated by the rings of the harness. As such, the electric current passes through the heart with little or no diminishment by the harness.

Various materials and methods can be used to coat the harness with dielectric material. In the illustrated embodiment, the rings are coated with silicone rubber. Other acceptable materials include Parylene™ and urethanes, as well as various polymers and the like. The materials can be applied to a harness by various methods, such as dip coating and spraying.

In the illustrated embodiment, the rings are placed on a mandrel and coated with dielectric material. It is contemplated that such a mandrel has an exterior surface configured such that the resulting ring structure is sized in conformity with the general anatomy of a human heart. Excess dielectric material is then removed from the cardiac harness, such that the shape of the dielectric material generally follows the shape of the spring members 140, as shown in FIG. 17. In this embodiment, excess dielectric material is left intact between some of the spring members of adjacent rings so as to provide nonconductive connectors 94 between adjacent rings. The excess dielectric material may be removed from the harness by using any cutting tool, such as a scalpel, laser, water jet, or the like.

As shown in FIG. 18, a connective junction 164 joins opposite ends 152 of each circumferentially extending ring 138. In one embodiment, the material including the dielectric material 162 secures the opposite ends of the rings. In another embodiment, the opposite ends of each ring may be further secured by applying silicone, or another similar material, before the dielectric material is applied to the harness. Also, the opposing ends may be welded, soldered, adhesively bonded, or held together by other means. In still another embodiment, the connective junctions may each have a small tube segment into which the opposite ends of the ring are inserted prior to application of the dielectric sheet to the harness. As discussed with reference to FIG. 15, the tube segment serves to prevent the opposite ends of the ring from tearing loose after the harness is placed on the heart. In this embodiment, the tube segments are covered with the dielectric material including the sheet of dielectric material 162.

A method of manufacturing a cardiac harness is now described with reference to FIGS. 19-21. The method generally includes configuring a metallic wire, and then covering the wire with an electrically insulative material. In one embodiment, Nitinol wire is first treated and shaped to develop a “remembered” shape having a harness portion 166 and a leader portion 168, as shown in FIG. 19. The harness portion has a plurality of spring members that are preferably arranged into a predefined configuration, such as an elongate strand 142. While held in the predefined configuration, the harness portion preferably is heat-set at a temperature of about 520° C. for about 20 minutes to establish the shape memory. The wire is then electropolished in accordance with standard methods known in the art. As shown in FIG. 19, the wire is configured such that the leader portion is disposed at one end of the harness portion of the wire.

Once the harness portion 166 of the wire is configured as described above, the wire is then covered with an electrically insulative material. In one embodiment, a tube of dielectric material is pulled over the wire. In a preferred embodiment, the tube is formed of silicone rubber. It will be appreciated that the inner diameter of the tube determines the level of tightness between the tube and wire. In one embodiment, wherein the wire has a diameter of about 0.012 inches, a silicone tube having an inner diameter of about 0.012 inches provides a relatively tight fit. In another embodiment, wherein the wire has a diameter of about 0.012 inches, a silicone tube having an inner diameter of about 0.020 inches provides a relatively loose fit. A silicone tube having an inner diameter smaller than the diameter of the wire can also be used to obtain a snug fit. In a preferred embodiment, silicone tubing sold under the trademark Nusil MED 4755 is used.

FIGS. 20 and 21 illustrate an apparatus and method for drawing a silicone rubber tube 170 over a harness portion 166, such as the portions illustrated in FIG. 19. The apparatus includes a clamp 172 into which one end of the harness portion is clamped. The leader portion 168 of the harness portion is preferably free. A pressure source 174 supplies solvent under a substantially constant pressure of, preferably, less than about 5 atmospheres and, more preferably, between about 1 to 2 atmospheres. The pressure source applies the solvent to a connector 176 which comprises a Y-shaped adapter. The solvent is supplied to one of the Y branches, another of the Y branches includes a compression valve 178, such as a Touhy-borst valve. Preferably, a hollow needle 180 extends from a base portion 182 of the Y adapter.

With particular reference to FIG. 20, the silicone tube 170 preferably is threaded over the outer diameter of the hollow needle 180. As such, solvent is supplied through the needle to the tube. In one embodiment, the solvent primarily includes a lubricant which facilitates sliding the tube over wire. Preferably, the lubricant is comprised of DOW OS-10, isopropyl alcohol (IPA), or another similar substance. In another embodiment, the solvent is a substance which primarily swells the inner diameter of the tube so as to facilitate sliding the tube over the wire. In such an embodiment, the solvent preferably includes hexane, heptane, xylene, and the like.

With continued reference to FIG. 20, once the solvent is flowing within and through the silicone tube, the free end of the leader portion 168 is threaded into the tube and the tube is advanced over the leader until the entire tube is disposed on the leader portion. With reference next to FIG. 21, once the leader portion has been advanced completely through the tube, the leader portion is threaded through the hollow needle and through the Touhy-borst valve 178 of the Y adapter. The free end of the leader portion is then clamped in place.

Due to the tortuous path defined by the spring elements 78 or 140, it may be difficult for the tubing 170 to be slid over the harness portion 166 without deforming the spring elements. However, in accordance with one embodiment, and with the assistance of the solvent, the tubing is drawn over the harness portion taking care not to substantially stretch the spring members. In accordance with another embodiment, the wire is pulled straight and held tightly in place between the clamps. In this manner, it is quite easy to advance the tubing over the harness portion because the spring elements of the harness portion have been substantially straightened out, as illustrated in FIG. 21. Once the tubing is disposed completely over the harness portion, the clamps 172 are released and, due to the shape memory and superelastic properties of Nitinol, the harness portion springs back substantially to its shape memory configuration. In accordance with a still further embodiment, once the free end of the leader has been clamped, the entire wire is stretched so that the spring elements of the harness portion are partially deformed, but are not stretched straight. In this manner, it becomes relatively easy to slide the tubing over the spring elements of the harness portion, but the spring elements are not deformed so much as to compromise their preformed memory shape. In this embodiment, care is taken to further deform the spring elements as little as possible while sliding the tubing into place.

In each of these embodiments, once the tube 170 has reached the end of the wire, and thus is covering the entire harness portion 166, the supply of pressurized solvent is stopped and the solvent supply apparatus is removed. The ends of the wire are removed from the clamps 172 and the leader portion 168 is trimmed from the harness portion. The harness portion substantially assumes its shape memory shape, and is ready to be further formed into a cardiac harness.

In order to relieve localized stresses that may exist between the tubing 170 and the wire, the tubing/wire combination is exposed to low level vibrations in order to help the tubing relax and shrink to a relaxed condition on the wire. In a preferred embodiment, the tubing/wire combination is treated with an ultrasonic cleaner which ultrasonically vibrates the combination. Such vibration can be termed “micromotion”, and helps the tubing and wire achieve a state of equilibrium relative to one another. As such, localized stresses that may have formed as the tubing was advanced over the wire are relaxed.

An important feature of the present invention is the minimally invasive delivery of the cardiac harness and the cardiac rhythm management device system which will be described immediately below.

Delivery of the cardiac harness 20,60, and 100 and associated electrodes and leads can be accomplished through conventional cardio-thoracic surgical techniques such as through a median sternotomy. In such a procedure, an incision is made in the pericardial sac and the cardiac harness can be advanced over the apex of the heart and along the epicardial surface of the heart simply by pushing it on by hand. The intact pericardium is over the harness and helps to hold it in place. The previously described grip pads and the compressive force of the cardiac harness on the heart provide sufficient attachment means of the cardiac harness to the epicardial surface so that sutures, clips or staples are unnecessary. Other procedures to gain access to the epicardial surface of the heart include making a slit in the pericardium and leaving it open, making a slit and later closing it, or making a small incision in the pericardium.

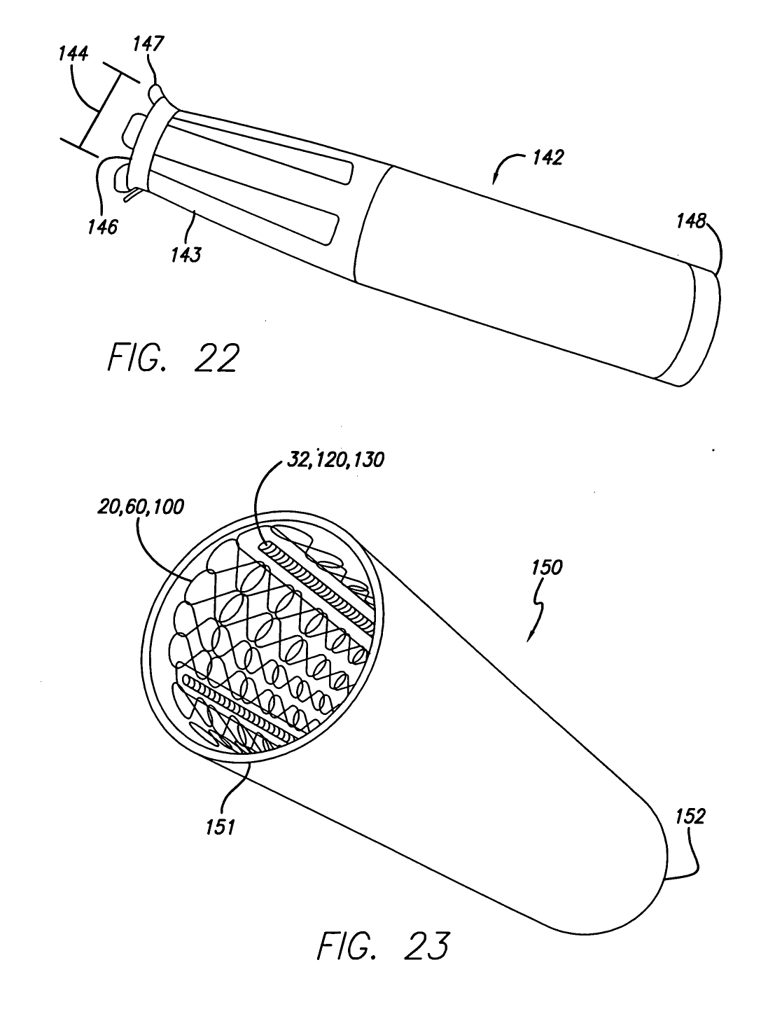

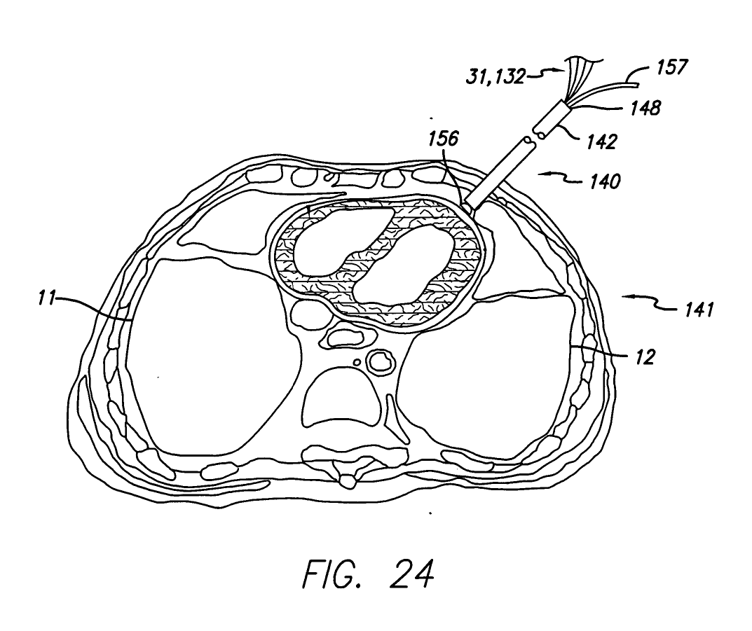

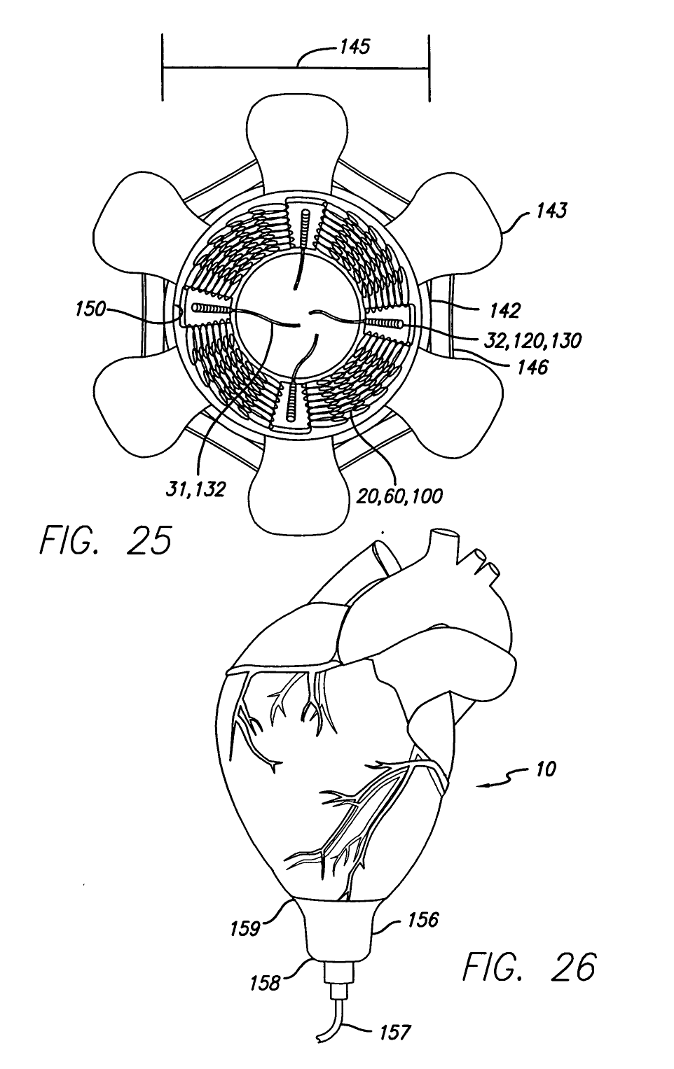

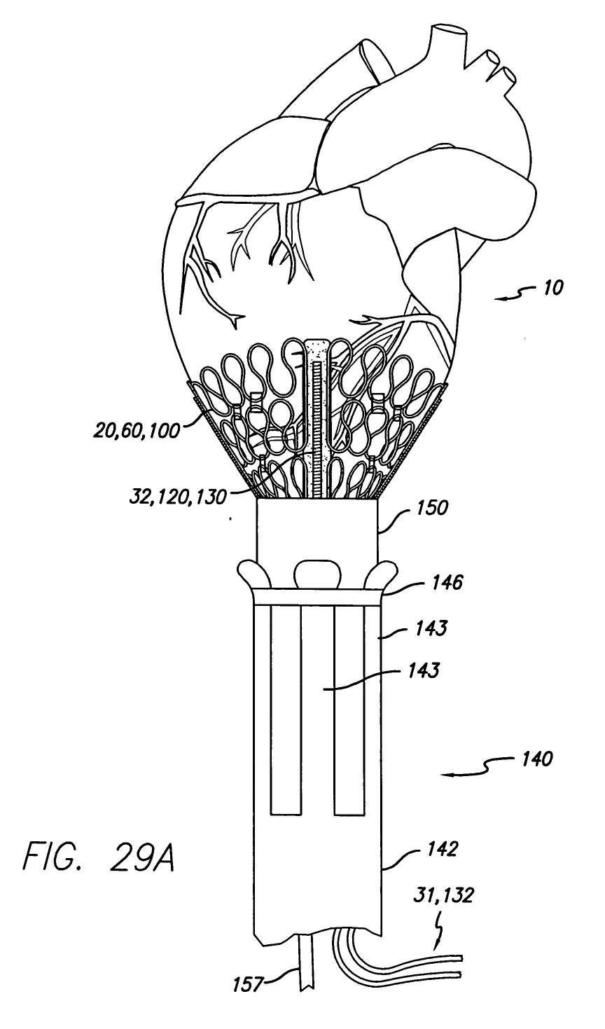

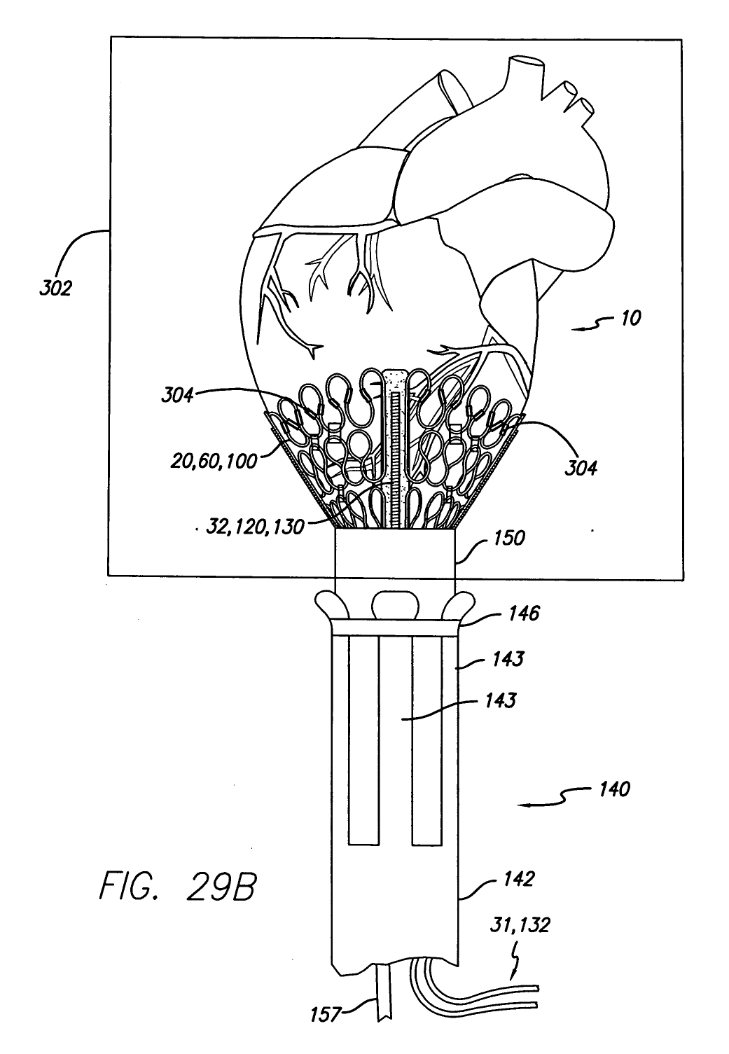

Preferably, however, the cardiac harness and associated electrodes and leads may be delivered through minimally invasive surgical access to the thoracic cavity, as illustrated in FIGS. 22-31B, and more specifically as shown in FIG. 24. A delivery device 140 may be delivered into the thoracic cavity 141 between the patient's ribs to gain direct access to the heart 10. Preferably, such a minimally invasive procedure is accomplished on a beating heart, without the use of cardio-pulmonary bypass. Access to the heart can be created with conventional surgical approaches. For example, the pericardium may be opened completely or a small incision can be made in the pericardium (pericardiotomy) to allow the delivery system 140 access to the heart. The delivery system of the disclosed embodiments comprises several components as shown in FIGS. 22-31B. As shown in FIG. 22, an introducer tube 142 is configured for low profile access through a patient's ribs. A number of fingers 143 are flexible and have a delivery diameter 144 as shown in FIG. 22, and an expanded diameter 145 as shown in FIG. 25. The delivery diameter is smaller than the expanded diameter. An elastic band 146 expands around the distal end 147 of the fingers and prevents the fingers from over expanding during delivery of the cardiac harness. The distal end of the fingers is the part of the delivery device 140 that is inserted through the patient's ribs to gain direct access to the heart.

The delivery device 140 also includes a dilator tube 150 that has a distal end 151 and a proximal end 152. The cardiac harness 20,60,100 is collapsed to a low profile configuration and inserted into the distal end of the dilator tube, as shown in FIG. 23. The dilator tube has an outside diameter that is slightly smaller than the inside diameter of the introducer tube 142. As will be discussed more fully herein, the distal end 151 of the dilator tube is inserted into the proximal end 147 of the introducer tube in close sliding engagement and in a slight frictional engagement. The slidable engagement between the dilator tube and the introducer tube should be with some mild resistance, however, there should be unrestricted slidable movement between the two tubes. The distal end 151 of the dilator tube will expand the fingers 143 of the introducer tube 142 as the dilator tube is pushed distally into the introducer tube as shown in FIG. 25. In the embodiments shown in FIGS. 22-31B, the cardiac harness 20, 60, 100 is equipped with leads (previously described) having electrodes for use in defibrillation or pacing functions.

As shown in FIG. 26, the delivery system 140 also includes a releasable suction device, such as suction cup 156 at the distal end of the delivery device. The negative pressure suction cup 156 is used to hold the apex of the heart 10. Negative pressure can be applied to the suction cup using a syringe or other vacuum device commonly known in the art. A negative pressure lock can be achieved by a one-way valve stop-cock or a tubing clamp, also known in the art. The suction cup 156 is formed of a biocompatible material and is preferably stiff enough to prevent any negative pressure loss through the heart while manipulating the heart and sliding the cardiac harness 20,60,100 onto the heart. Further, the suction cup 156 can be used to lift and maneuver the heart 10 to facilitate advancement of the harness or to allow visualization and surgical manipulation of the posterior side of the heart. The suction cup has enough negative pressure to allow a slight pulling in the proximal direction away from the apex of the heart to somewhat elongate the heart (e.g., into a bullet shape) during delivery to facilitate advancing the cardiac harness over the apex and onto the base portion of the heart. After the suction cup 156 is attached to the apex of the heart and a negative pressure is drawn, the cardiac harness, which has been releasably mounted in the distal end 151 of the dilator tube 150, can be advanced distally over the heart, as will be described more fully herein.

As shown in FIG. 24, the delivery device 140, and more specifically introducer tube 142, has been advanced through the intercostal space between the patient's ribs during insertion of the introducer tube, the fingers 143 are in their delivery diameter 144, which is a low profile for ease of access through the small port made through the patient's ribs. Thereafter, the dilator tube 150, with the cardiac harness 20,60,100 mounted therein, is advanced distally through the introducer tube so that the fingers 143 are expanded until they achieve their expanded diameter 145. The suction cup 156 can be attached to the apex 13 of the heart 10 either before or after the dilator tube is advanced to spread the fingers 143 of the introducer tube 142. Preferably, the dilator tube has already expanded the fingers on the introducer tube so that there is a larger opening for the suction cup as it is advanced through the inside of a dilator tube, out of the distal end of the introducer tube, and placed in contact with the apex of the heart. Thereafter, a negative pressure is drawn allowing the suction cup to securely attach to the apex of the heart. Visualizing equipment that is commonly known in the art may be used to assist in positioning the suction cup to the apex. For example, fluoroscopy, magnetic resonance imaging (MRI), dye injection to enhance fluoroscopy, and echocardiography, and intracardiac, transesophageal, or transthoracic echo, all can be used to enhance positioning and in attaching the suction cup to the apex of the heart. After negative pressure is drawn and the suction cup is securely attached (releasably) to the apex of the heart, the heart can then be maneuvered somewhat by pulling on the tubing 157 attached to the suction cup, or by manipulating the introducer tube 142, the dilator tube 150, both in conjunction with the suction cup. As previously described, it may be advantageous to pull on the tubing 157 to allow the suction cup to pull on the apex of the heart and elongate the heart somewhat in order to facilitate sliding the harness over the epicardium.

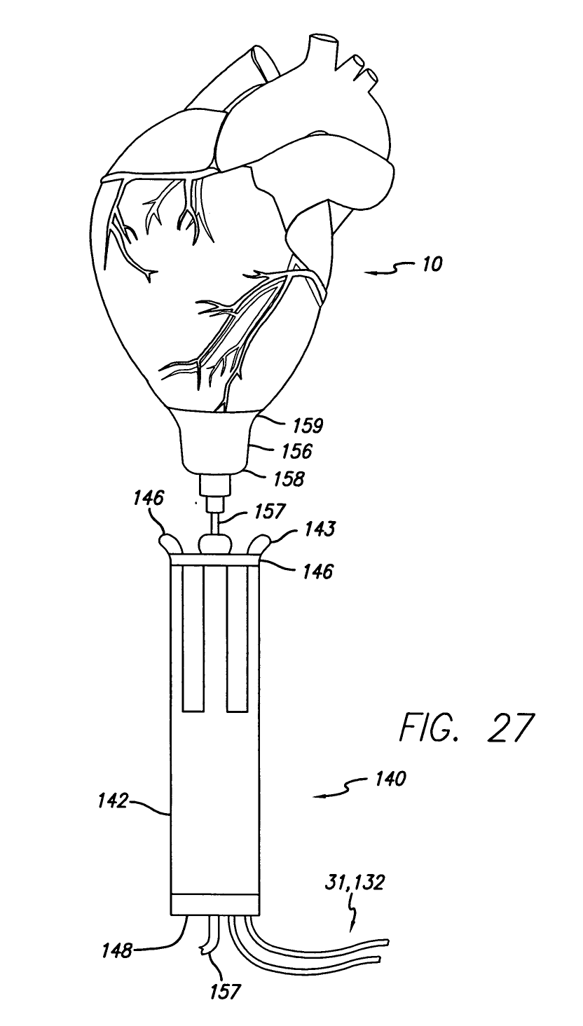

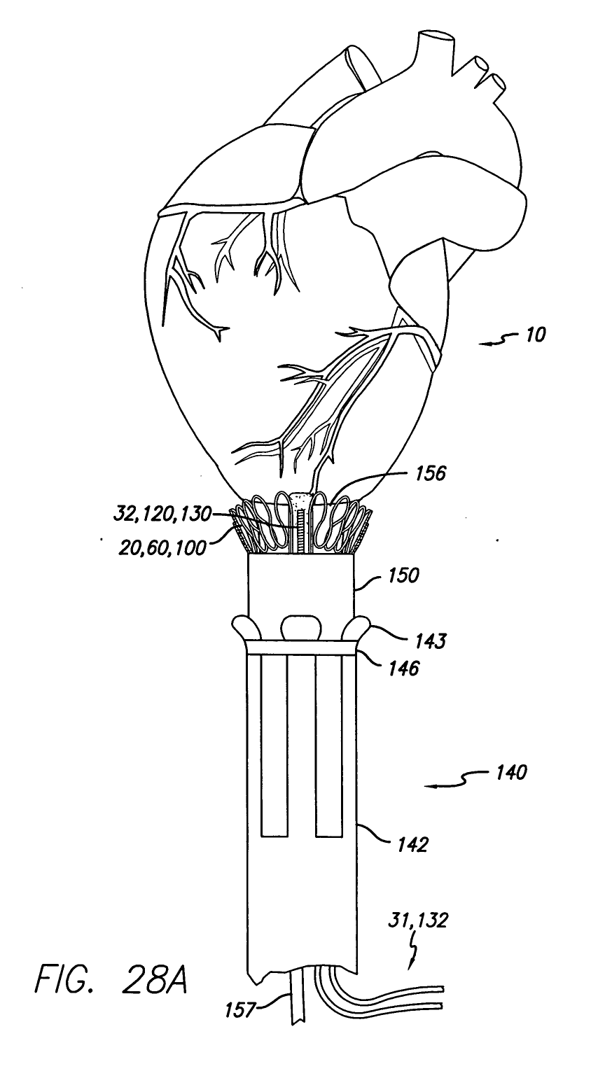

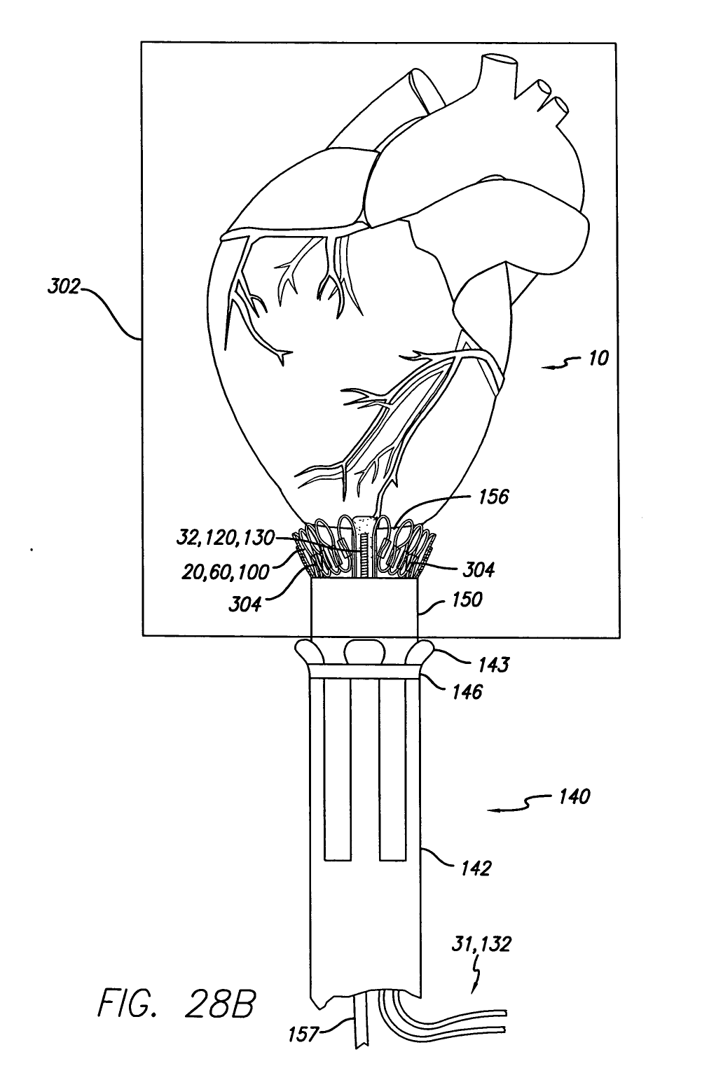

As more clearly shown in FIGS. 27-31B, the cardiac harness 20,60,100 is advanced distally out of the dilator tube and over the suction cup 156. The suction cup is tapered so that the distal end of the harness slides over the narrow portion of the taper (the proximal end of the suction cup 158). The suction cup becomes wider at its distal end where it is attached to the apex of the heart, and the cardiac harness continues to slide and expand over the suction cup as it is advanced distally. As the cardiac harness continues to be advanced distally, it slides over the apex of the heart and continues to expand as it is pushed out of the dilator tube and along the epicardial surface of the heart. Since the harness and the electrodes 32,120,130 are coated with the previously described dielectric material, preferably silicone rubber, the cardiac harness should slide easily over the epicardial surface of the heart. The silicone rubber offers little resistance and the epicardial surface of the heart has sufficient fluid to allow the harness to easily slide over the wet surface of the heart. The pericardium previously has been cut so that the cardiac harness is sliding over the epicardial surface of the heart with the pericardium over the cardiac harness to help hold it onto the surface of the heart. In further keeping with the invention, the previously described cardiac harness 20,60,100 has a polymer coating that increases the radiopacity of the cardiac harness. Similarly, the cardiac harness can have previously described radiopaque bands to increase the radiopacity of the cardiac harness. With reference to FIGS. 28B, 29B, 30B and 31B, the cardiac harness 20,60,100 has a radiopaque coating 300 as shown by the bold lines in the drawings. The radiopaque coating is the result of the polymer coating surrounding the cardiac harness being loaded with a radiopaque material such as BaSO₄, or similar material, as previously described. X-ray device 302 provides a fluoroscopic image of the cardiac harness in which the radiopaque coating 300 is clearly visible, and is substantially more radiopaque than the remaining portion of the cardiac harness that does not have a radiopaque coating. Alternatively, the radiopacity of the cardiac harness 20,60,100 can be enhanced by radiopaque bands 304 as previously described. The number of radiopaque bands 304, or radiopaque coating 300 is a matter of choice as previously described.

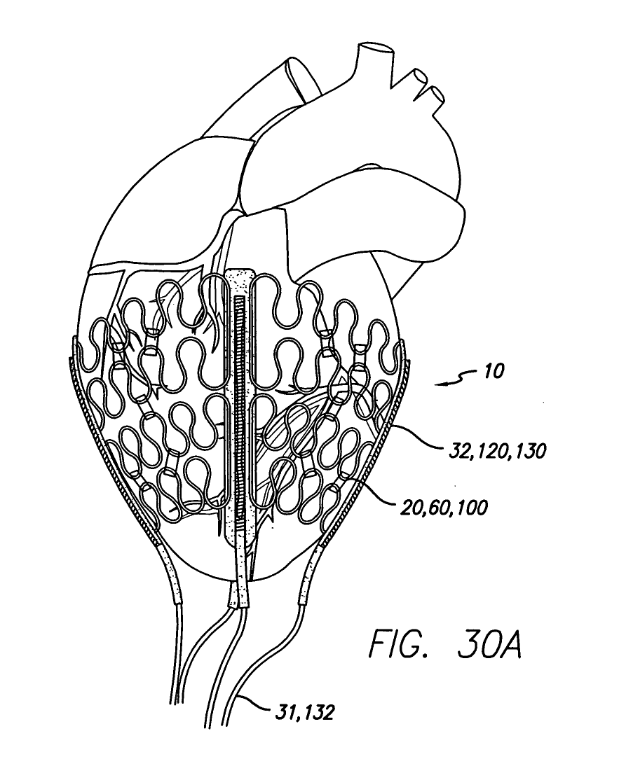

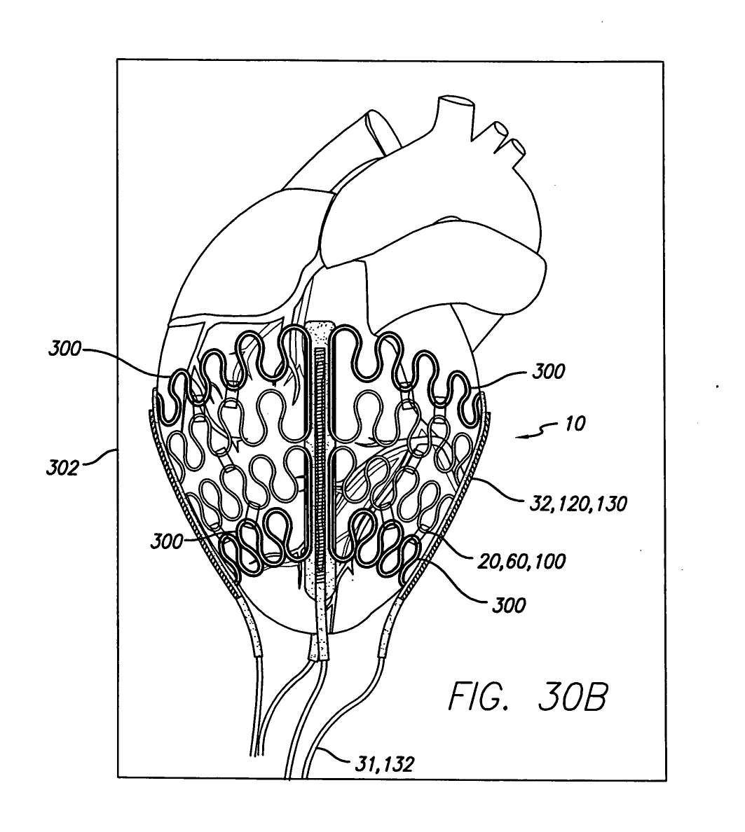

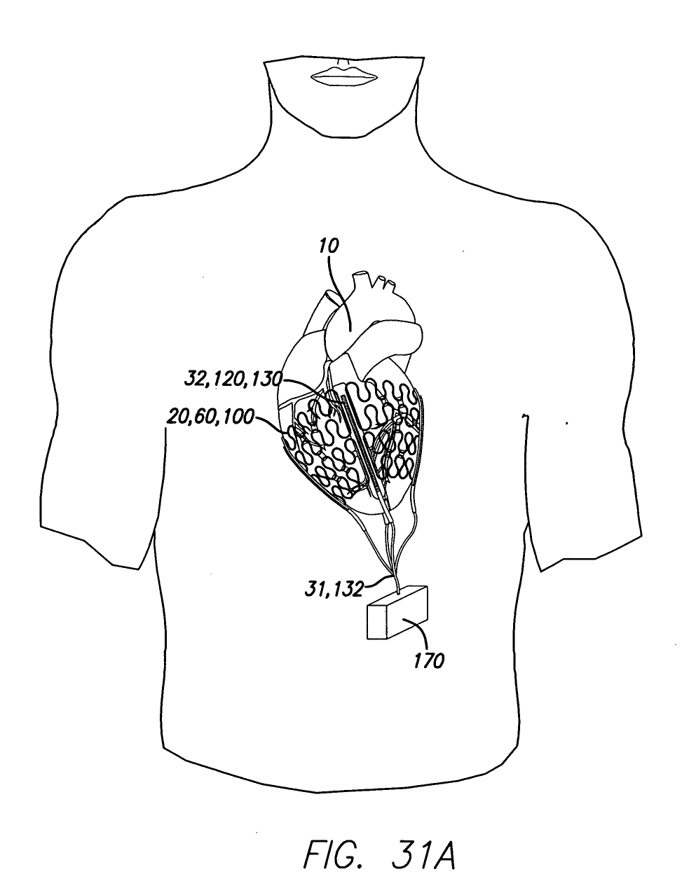

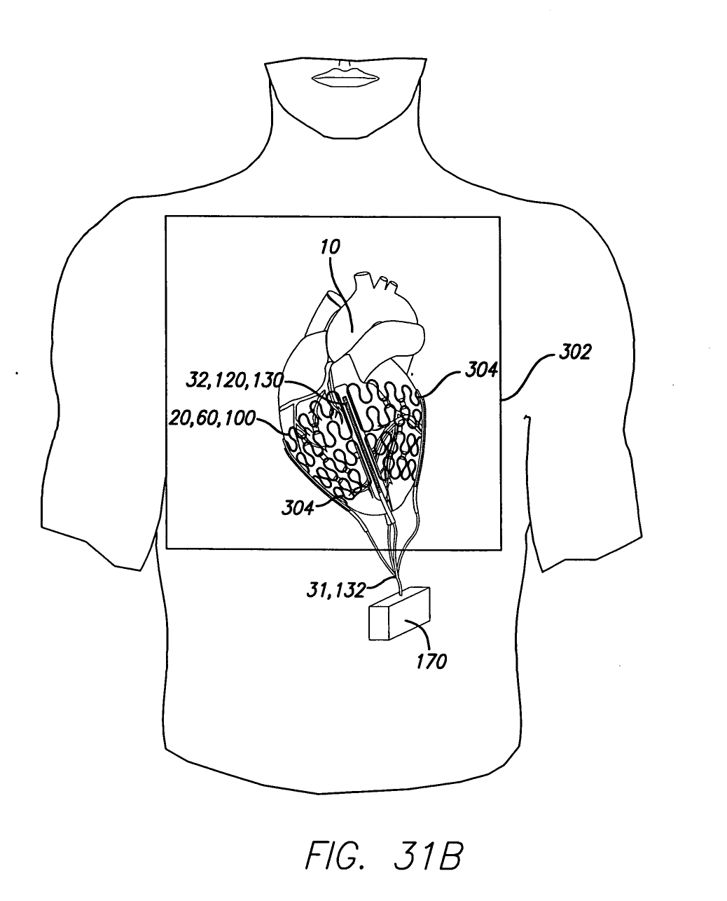

As shown in FIGS. 30A, 30B, 31A, and 31B, the cardiac harness 20,60,100 has been completely advanced out of the dilator tube so that the harness covers at least a portion of the heart 10. The suction cup 156 has been withdrawn, and the introducer tube 142 and dilator tube 150 also have been withdrawn proximally from the patient. Prior to removing the introducer tube, a power source 170 (such as an ICD, CRT-D, and/or pacemaker) can be implanted by conventional means. The electrodes will be attached to the pulse generator to provide a defibrillating shock or pacing functions as previously described.

In the embodiments shown in FIGS. 22-31B, the cardiac harness 20,60,100 was advanced through the dilator tube by pushing on the proximal end of the electrodes 32,120,130, on the lead wires 31,133, and on the proximal end (apex 26) of the cardiac harness. Even though the electrodes are designed to be atraumatic and longitudinally flexible, the electrodes have sufficient column strength so that pushing on the proximal ends of the electrodes assists in pushing the cardiac harness out of the dilator tube and over the epicardial surface of the heart. In one embodiment, advancement of the cardiac harness is accomplished by hand, by the physician simply pushing on the electrodes and the leads to advance the cardiac harness out of the dilator tube to slide onto the epicardial surface of the heart.

As shown in the embodiments of FIGS. 22-31B, the delivery device 140, and more specifically introducer tube 142 and dilator tube 150, have a circular cross-section. It may be preferable, however, to chose other cross-sectional shapes, such as an oval cross-sectional shape for the delivery device. An oval delivery device may be more easily inserted through the intercostal space between the patient's ribs for a low profile delivery. Further, as the cardiac harness 20,60,100 is advanced out of a delivery device 140 having an oval cross-section, the harness distal end will quickly form into a more circular shape in order to assume the configuration of the epicardial surface of the heart as it is advanced distally over the heart.

In the embodiments shown in FIGS. 30A, 30B, 31A, and 31B, the cardiac harness 20,60,100 remains firmly attached to the epicardial surface of the heart without the need for any further attachment means, such as sutures, clips, adhesives, or staples. Further, the pericardial sac helps to enclose the harness to prevent it from shifting or sliding on the epicardial surface of the heart.

It may be desired to reduce the likelihood of the development of fibrotic tissue over the cardiac harness so that the elastic properties of the harness are not compromised. Also, as fibrotic tissue forms over the cardiac harness and electrodes over time, it may become necessary to increase the power of the pacing stimuli. As fibrotic tissue increases, the right and left ventricular thresholds may increase, commonly referred to as “exit block.” When exit block is detected, the pacing therapy may have to be adjusted. Certain drugs such as steroids, have been found to inhibit cell growth leading to scar tissue or fibrotic tissue growth. Examples of therapeutic drugs or pharmacologic compounds that may be loaded onto the cardiac harness or into a polymeric coating on the harness, on a polymeric sleeve, on individual undulating strands on the harness, or infused through the lumens in the electrodes and delivered to the epicardial surface of the heart include steroids, taxol, aspirin, prostaglandins, and the like. Various therapeutic agents such as antithrombogenic or antiproliferative drugs are used to further control scar tissue formation. Examples of therapeutic agents or drugs that are suitable for use in accordance with the present invention include 17-beta estradiol, sirolimus, everolimus, actinomycin D (ActD), taxol, paclitaxel, or derivatives and analogs thereof. Examples of agents include other antiproliferative substances as well as antineoplastic, antiinflammatory, antiplatelet, anticoagulant, antifibrin, antithrombin, antimitotic, antibiotic, and antioxidant substances. Examples of antineoplastics include taxol (paclitaxel and docetaxel). Further examples of therapeutic drugs or agents include antiplatelets, anticoagulants, antifibrins, antiinflammatories, antithrombins, and antiproliferatives. Examples of antiplatelets, anticoagulants, antifibrins, AND antithrombins include, but are not limited to, sodium heparin, low molecular weight heparin, hirudin, argatroban, forskolin, vapiprost, prostacyclin and prostacyclin analogs, dextran, D-phe-pro-arg-chloromethylketone (synthetic antithrombin), dipyridamole, glycoprotein IIb/IIIa platelet membrane receptor antagonist, recombinant hirudin, thrombin inhibitor (available from Biogen located in Cambridge, Mass.), and 7E-3B® (an antiplatelet drug from Centocor located in Malvern, Pa.). Examples of antimitotic agents include methotrexate, azathioprine, vincristine, vinblastine, fluorouracil, adriamycin, and mutamycin. Examples of cytostatic or antiproliferative agents include angiopeptin (a somatostatin analog from Ibsen located in the United Kingdom), angiotensin converting enzyme inhibitors such as Captopril® (available from Squibb located in New York, N.Y.), Cilazapril® (available from Hoffman-LaRoche located in Basel, Switzerland), or Lisinopril® (available from Merck located in Whitehouse Station, N.J.); calcium channel blockers (such as Nifedipine), colchicine, fibroblast growth factor (FGF) antagonists, fish oil (omega 3-fatty acid), histamine antagonists, Lovastatin® (an inhibitor of HMG-CoA reductase, a cholesterol lowering drug from Merck), methotrexate, monoclonal antibodies (such as PDGF receptors), nitroprusside, phosphodiesterase inhibitors, prostaglandin inhibitor (available from GlaxoSmithKline located in United Kingdom), Seramin (a PDGF antagonist), serotonin blockers, steroids, thioprotease inhibitors, triazolopyrimidine (a PDGF antagonist), and nitric oxide. Other therapeutic drugs or agents which may be appropriate include alpha-interferon, genetically engineered epithelial cells, and dexamethasone.

Although the present invention has been described in terms of certain preferred embodiments, other embodiments that are apparent to those of ordinary skill in the art are also within the scope of the invention. Accordingly, the scope of the invention is intended to be defined only by reference to the appended claims. While the dimensions, types of materials and coatings described herein are intended to define the parameters of the invention, they are by no means limiting and are exemplary embodiments.

1. A cardiac harness, comprising:
a plurality of hinge elements formed in circumferential rows;
a plurality of connectors for connecting adjacent rows together;
at least a portion of the rows being coated with a flexible polymer; and
the polymer being loaded with a radiopaque material so that the radiopaque material is visible under fluoroscopy.

2. The cardiac harness of claim 1, wherein the flexible polymer is taken from the group of polymers consisting of silicone rubber, Parylene®, urethanes, polyethylene, polypropylene, polyurethane, nylon, PTFE and ePTFE.

3. The cardiac harness of claim 2, wherein the radiopaque material is taken from the group of materials consisting of barium sulfate, barium, tungsten, bismuth subcarbonate, bismuth trioxide, bismuth oxychloride, tantalum, iridium, gold, silver, platinum, or other metals having high mass density.

4. The cardiac harness of claim 3, wherein the radiopaque material is loaded into the polymer in the range of about 5% up to about 40% by weight.

5. The cardiac harness of claim 3, wherein the radiopaque material is in the form of a powder.

6. The cardiac harness of claim 1, wherein a base row is coated with the radiopaque polymer.

7. The cardiac harness of claim 1, the radiopaque material being visible under MRI, echocardiography and intracardiac, transesophageal, or transthoracic echocardiography.

8. The cardiac harness of claim 1, wherein at least some of the connectors are formed from a polymer loaded with a radiopaque material.

9. The cardiac harness of claim 1, wherein the cardiac harness further comprises electrodes associated with a power source.

10. The cardiac harness of claim 9, wherein a dielectric material electrically insulates the cardiac harness from the electrodes at least a portion of the dielectric material being loaded with a radiopaque material.

11. A cardiac harness, comprising:
a plurality of hinge elements formed in circumferential rows;
a plurality of connectors arranged to connect adjacent rows together;
a plurality of radiopaque bands attached to the hinge elements.

12. The cardiac harness of claim 11, wherein the bands are formed from a metal or metal alloy taken from the group consisting of platinum, gold, platinum iridium, tantalum, tungsten, Parylene™, urethane, polytetrafluoroethylene or an elastomer.

13. The cardiac harness of claim 11, wherein the radiopaque bands are crimped onto the hinge elements.

14. The cardiac harness of claim 13, wherein the radiopaque bands are crimped onto a straight portion of the hinge elements.

15. The cardiac harness of claim 11, wherein the radiopaque bands are plated onto the hinge elements.

16. The cardiac harness of claim 11, wherein the radiopaque bands are coated onto the hinge elements by sputter coating or ion plasma deposition.

17. The cardiac harness of claim 11, wherein the radiopaque bands are positioned on a base row.

18. The cardiac harness of claim 11, wherein the radiopaque bands are positioned on an apex row.

19. A method for mounting a cardiac harness on a heart, comprising:
providing a cardiac harness having at least a portion coated with a radiopaque material;
advancing the cardiac harness onto the heart;
visualizing the radiopaque material so that the position of the cardiac harness can be monitored as the cardiac harness is mounted onto the heart.

20. The method of claim 19, wherein the cardiac harness is coated with a polymer loaded with a radiopaque material.

21. The method of claim 20, wherein the radiopaque material is loaded into the polymer in the range of about 5% up to about 40% by weight.

22. The method of claim 21, wherein the radiopaque material is taken from the group consisting of barium sulfate, barium, tungsten, bismuth subcarbonate, bismuth trioxide, bismuth oxycholoride, tantalum, iridium, gold, silver, platinum or other metals of high mass density.

23. The method of claim 19, wherein visualizing the radiopaque material further comprises utilizing fluoroscopy, MRI, echocardiography, intracardiac, transesophageal, or transthoracic echocardiography.
